(19) United States
(12) Patent Application Publication
(10) Pub. No.: US 2025/0387089 A1
(43) Pub. Date: Dec. 25, 2025

(54) X-Ray Imaging Method, X-Ray Imaging System, and X-Ray Imaging Program

(71) Applicant: SHIMADZU CORPORATION, Kyoto-shi (JP)

(72) Inventors: Junya YAMAMOTO, Kyoto-shi (JP); Hiroshi OKUMURA, Kyoto-shi (JP); Ryo TAKEDA, Kyoto-shi (JP)

(21) Appl. No.: 19/127,348

(22) PCT Filed: Sep. 15, 2023
(86) PCT No.: PCT/JP2023/033729

§ 371 Date: May 5, 2025

(30) Foreign Application Priority Data
Dec. 27, 2022 (JP) 2022-210025

(51) Int. Cl. A61B 6/00; A61B 6/46; A61B 6/50

(52) U.S. Cl. CPC A61B 6/488; A61B 6/461; A61B 6/469; A61B 6/505

(57) ABSTRACT

An X-ray imaging method according to this invention includes acquiring, in a mobile terminal 4, a pre-captured X-ray image Gx1 captured through X-rays with which a subject Pa is irradiated from an X-ray irradiator 11 and that pass through the subject; acquiring, in the mobile terminal 4, imaging assistance information J for assistance in capturing a main captured X-ray image Gx2 captured through X-rays with which the subject is irradiated from the X-ray irradiator 11 based on the pre-captured X-ray image Gx1 after capturing the pre-captured X-ray image Gx1; and displaying the imaging assistance information J on a display 44 of the mobile terminal 4 before capturing the main captured X-ray image Gx2.

The present invention relates to an X-ray imaging method, an X-ray imaging system and an X-ray imaging program, particularly to an X-ray imaging method, an X-ray imaging system and an X-ray imaging program irradiating a subject with X-rays from an X-ray irradiator.

### BACKGROUND ART

X-ray imaging systems irradiating a subject with X-rays from an X-ray irradiator are known in the art (see, for example, Patent Document 1).

The above Patent Document 1 discloses a radiation imaging apparatus irradiating a subject with X-rays from an X-ray irradiator. This radiation imaging apparatus includes an operation block (X-ray irradiator) irradiating the subject with X-rays. The operation block includes an X-ray tube and a handle. The X-ray tube is configured to irradiate the subject with X-rays. The handle is configured to be gripped by an operator when the operator moves the operation block in horizontal directions and an upward/downward direction. In the radiation imaging apparatus, when irradiating the subject with X-rays to capture X-ray images, a position of the X-ray tube is adjusted by the operator operating the handle.

### PRIOR ART

### Patent Document

Patent Document 1: Japanese Patent Laid-Open Publication No. JP 2010-227376

### SUMMARY OF THE INVENTION

### Problems to be Solved by the Invention

Here, although not explicitly stated in the above Patent Document 1, in such a known radiation imaging apparatus as disclosed in the above Patent Document 1, a display displaying X-ray images of the subject captured after adjustment of the position of the X-ray tube is arranged outside of an imaging room. In this case, it is conceived that the efficiency of the operator's imaging work may deteriorate when capturing accurately positioned X-ray images. Here, as capture of the accurately positioned X-ray images, capture of X-ray images of a side of a knee joint is provided for diagnosing diseases around the knee joint, such as osteochondritis dissecans and knee osteoarthritis. In this capture of X-ray images, it is necessary to capture an X-ray image in which an outer edge of a medial condyle and an outer edge of a lateral condyle of a femur. However, in capture of X-ray images of the side of the knee joint, it is difficult for even experienced operators (radiologic technologist) to capture the accurately positioned X-ray images.

For this reason, when capturing the above images by using the known radiation imaging apparatus as disclosed in the above Patent Document 1, if the operator is not experienced, repeated adjustment of the position of the X-ray tube after checking X-ray images displayed on the display, which is arranged outside of the imaging room, increases the number of operations of the operator. In this case, the efficiency of the operator's imaging work deteriorates when capturing the X-ray images. Accordingly, if the operator (user) is not experienced, the operator may not efficiently capture the X-ray images necessary for accurate diagnosis of the disease.

The present invention is intended to solve the above problem, and one object of the present invention is to provide an X-ray imaging method, an X-ray imaging system and an X-ray imaging program allowing an operator to efficiently capture X-ray images necessary for accurate diagnosis of diseases even if the user is not experienced.

### Means for Solving the Problems

In order to attain the aforementioned object, an X-ray imaging method according to a first aspect of the present invention includes acquiring, in a mobile terminal, a pre-captured X-ray image captured through X-rays with which a subject is irradiated from an X-ray irradiator and that pass through the subject; acquiring, in the mobile terminal, imaging assistance information for assistance in capturing a main captured X-ray image captured through X-rays with which the subject is irradiated from the X-ray irradiator based on the pre-captured X-ray image after capturing the pre-captured X-ray image; and displaying the imaging assistance information on a display of the mobile terminal before capturing the main captured X-ray image.

Also, an X-ray imaging system according to a second aspect of the present invention includes an X-ray imaging apparatus including an X-ray irradiator irradiating a subject with X-rays and an X-ray detector detecting X-rays with which the subject is irradiated from the X-ray irradiator and that pass through the subject; and a mobile terminal provided separately from the X-ray imaging apparatus, wherein the mobile terminal includes a controller acquiring imaging assistance information for assistance in capturing a main captured X-ray image captured through X-rays with which the subject is irradiated from the X-ray irradiator based on a pre-captured X-ray image captured through X-rays with which the subject is irradiated from the X-ray irradiator and that pass through the subject after capturing the pre-captured X-ray image, and a display configured to display the imaging assistance information acquired in the controller.

Also, an X-ray imaging program according to a third aspect of the present invention is an X-ray imaging program to be executed in a mobile terminal, the program causing the mobile terminal to perform acquiring a pre-captured X-ray image captured through X-rays with which a subject is irradiated from an X-ray irradiator and that pass through the subject; acquiring imaging assistance information for assistance in capturing a main captured X-ray image captured through X-rays with which the subject is irradiated from the X-ray irradiator based on the pre-captured X-ray image after capturing the pre-captured X-ray image; and displaying the imaging assistance information on a display of the mobile terminal before capturing the main captured X-ray image.

### Effect of the Invention

In the X-ray imaging method according to the aforementioned first aspect, the X-ray imaging system according to the aforementioned second aspect, and the X-ray imaging program according to the aforementioned third aspect, as discussed above, imaging assistance information for assistance in capturing a main captured X-ray image captured through X-rays with which the subject is irradiated from the X-ray irradiator based on the pre-captured X-ray image is acquired after capturing the pre-captured X-ray image in the mobile terminal. In addition, the imaging assistance information is displayed on a display of the mobile terminal before capturing the main captured X-ray image. Accordingly, users can adjust a position of the X-ray irradiator, its dose and the like while checking the mobile terminal displaying the imaging assistance information on the spot. Consequently, even if the users are not experienced, it is possible to prevent repeated adjustment of the X-ray irradiator after checking X-ray images displayed on a display arranged outside of an imaging room. Also, the users can acquire appropriate imaging assistance information in the mobile terminal by acquiring imaging assistance information based on the pre-captured X-ray image. As these results, even if the users are not experienced, the users can efficiently capture the X-ray images necessary for accurate diagnosis of diseases.

### BRIEF DESCRIPTION OF THE DRAWINGS

FIG. 1 is a side view showing an X-ray imaging system according to a first embodiment.

FIG. 2 is a block diagram showing a configuration of an X-ray imaging apparatus of the X-ray imaging system according to the first embodiment.

FIG. 3 is a block diagram showing a configuration of a mobile terminal of the X-ray imaging system according to the first embodiment.

FIG. 4 is a perspective view showing a state for capturing an X-ray image of a patient through the X-ray imaging system according to the first embodiment.

FIG. 5 is a schematic view showing a pre-captured X-ray image captured through the X-ray imaging apparatus of the X-ray imaging system according to the first embodiment.

FIG. 6 is a schematic view showing a main captured X-ray image captured through the X-ray imaging apparatus of the X-ray imaging system according to the first embodiment.

FIG. 7 is a schematic view showing a display of the mobile terminal of the X-ray imaging system according to the first embodiment with imaging assistance information being indicated.

FIG. 8 is a schematic view showing a method for estimating an outer edge of a lateral condyle and an outer edge of a medial condyle in the mobile terminal of the X-ray imaging system according to the first embodiment.

FIG. 9 is a schematic view showing the display of the mobile terminal of the X-ray imaging system according to the first embodiment with a screen for confirming whether to acquire position correction information being indicated on the display.

FIG. 10 is a graph showing a relation between a deviation amount and a position of an X-ray tube used when the position correction information is acquired in the mobile terminal of the X-ray imaging system according to the first embodiment.

FIG. 11 is a schematic view showing the display of the mobile terminal of the X-ray imaging system according to the first embodiment with a center position of a light beam emitted for irradiation from a collimator lamp of the X-ray imaging apparatus being aligned with a base end of an arrow indicated on the display of the mobile terminal.

FIG. 12 is a schematic view showing the display of the mobile terminal of the X-ray imaging system according to the first embodiment with a center position of a light beam emitted for irradiation from the collimator lamp of the X-ray imaging apparatus being aligned with a head end of the arrow indicated on the display of the mobile terminal.

FIG. 13 is a flowchart illustrating an X-ray imaging method performed in the X-ray imaging system according to the first embodiment.

FIG. 14 is a side view showing an X-ray imaging system according to a second embodiment with a pre-captured X-ray image displayed on a display of an image processing apparatus is captured through an image capturer of a mobile terminal.

FIG. 15 is a side view showing the X-ray imaging system according to the second embodiment with a radiologic technologist being adjusting an X-ray irradiator while watching the mobile terminal.

FIG. 16 is a flowchart illustrating an X-ray imaging method performed in the X-ray imaging system according to the second embodiment.

FIG. 17 is a side view showing an X-ray imaging system according to a third embodiment with a pre-captured X-ray image stored in an image processing apparatus being stored through a portable storage medium.

FIG. 18 is a side view showing the X-ray imaging system according to the third embodiment with a radiologic technologist being adjusting an X-ray irradiator while watching the mobile terminal.

FIG. 19 is a flowchart illustrating an X-ray imaging method performed in the X-ray imaging system according to the third embodiment.

FIG. 20 is a side view showing an X-ray imaging system according to a fourth embodiment.

FIG. 21 is a block diagram showing a configuration of an X-ray imaging apparatus of the X-ray imaging system according to the fourth embodiment.

FIG. 22 is a view showing a collimator of the X-ray imaging apparatus of the X-ray imaging system according to the fourth embodiment as viewed from a lower side.

FIG. 23 is a schematic view showing a state before an X-ray irradiation device of the X-ray imaging system according to the fourth embodiment arrives at a corrected position.

FIG. 24 is a schematic view showing a state in which the X-ray irradiation device of the X-ray imaging system according to the fourth embodiment has arrived at the corrected position.

FIG. 25 is a flowchart illustrating an X-ray imaging method performed in the X-ray imaging system according to the fourth embodiment.

### MODES FOR CARRYING OUT THE INVENTION

The following description will describe embodiments embodying the present invention with reference to the drawings.

### First Embodiment

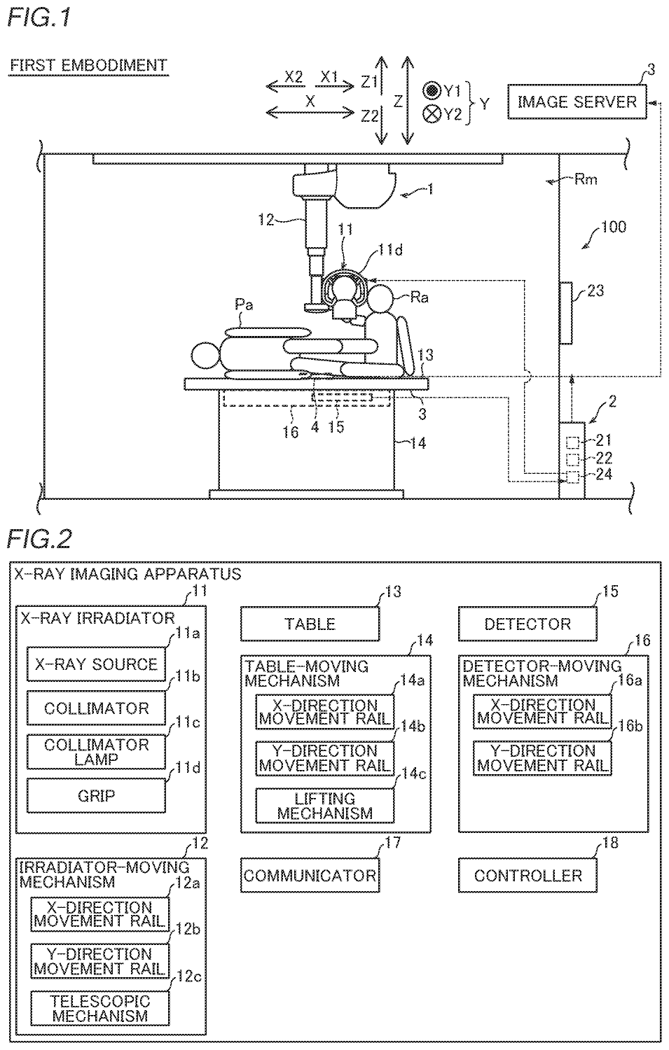

The following description first describes a configuration of an X-ray imaging system 100 according to a first embodiment of the present invention with reference to FIGS. 1 to 13.

As shown in FIGS. 1 and 2, the X-ray imaging system 100 is a system for performing general examinations using X-rays conducted in initial diagnosis of a patient Pa who visits a medical facility such as a hospital or clinic. Here, the patient Pa is an example of a “subject” in the claims.

Specifically, the X-ray imaging system 100 includes an X-ray imaging apparatus 1, an image processing apparatus 2, an image server 3, and a mobile terminal 4. In the X-ray imaging system 100, a network is constructed to allow the X-ray imaging apparatus 1 and the image processing apparatus 2 to communicate with each other. In the X-ray image capture system 100, the network is constructed to allow the image processing apparatus 2, the image server 3, and the mobile terminal 4 to communicate with each other.

### (X-Ray Imaging Apparatus)

The X-ray imaging apparatus 1 is an apparatus for capturing X-ray images Gx of the patient Pa by using X-rays. The X-ray imaging apparatus 1 includes an X-ray irradiator 11, an irradiator-moving mechanism 12, a table 13, a table-moving mechanism 14, a detector 15, a detector-moving mechanism 16, a communicator 17, and a controller 18.

Here, an upward/downward direction is defined as a Z direction, and upward and downward directions are defined as Z1 and Z2 directions, respectively. In horizontal directions, longitudinal directions of the table 13 is defined as X directions, and one direction (a direction toward the image processing apparatus 2 side in FIG. 1) of the X directions and another direction (a direction toward a side opposite to the image processing apparatus 2 side in FIG. 1) of the X directions are defined as an X1 direction and an X2 direction, respectively. In horizontal directions, directions perpendicular to the X directions are defined as Y directions (shorter directions of the table 13), and one direction of the Y directions and another direction of the Y directions are defined as a Y1 direction and a Y2 direction, respectively.

In the following description, it is assumed that the X-ray irradiator 11, the table 13, and the detector 15 are all configured to be manually moved. Manually refers to that a radiologic technologist Ra moves the X-ray irradiator 11, the table 13, and the detector 15 by pressing or pulling on their respective parts.

Also, it is assumed that the X-ray imaging apparatus 1 includes no potentiometers corresponding to positions in the XY directions (horizontal directions), a position of the Z direction (upward/downward direction), a rotational angle position about a rotation axis extending in the Z direction, and rotational angle positions of a plurality of predetermined rotation axes extending in directions perpendicular to the Z direction when each of the X-ray irradiator 11, the table 13, and the detector 15 is manually moved.

### <X-Ray Irradiator>

The X-ray Irradiator 11 is configured to irradiate the patient Pa with X-rays as shown in FIG. 2. Specifically, the X-ray irradiator 11 includes an X-ray source 11a, a collimator 11b, a collimator lamp 11c, and a grip 11d.

The X-ray source 11a is an X-ray tube that generates X-rays when a high voltage is applied.

The collimator 11b is configured to adjust an irradiation direction and an irradiation area of X-rays generated in the X-ray source 11a.

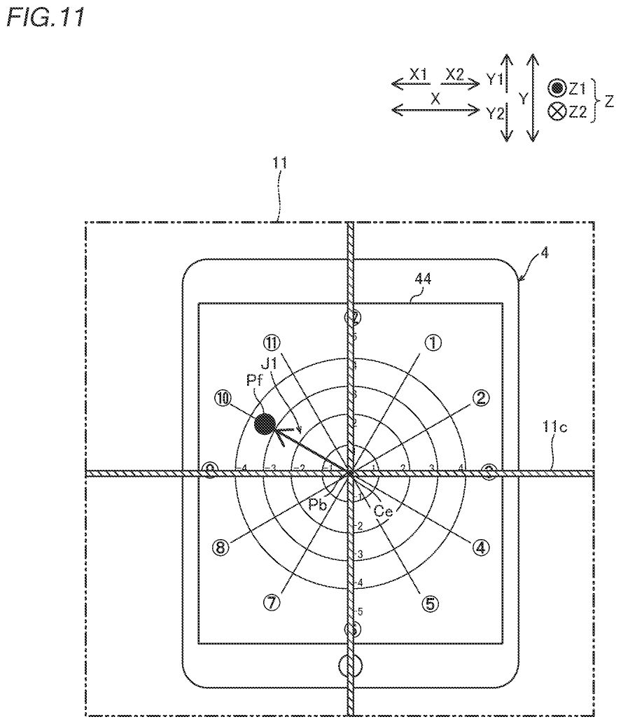

The collimator lamp 11c includes an LED (Light Emitting Diode) that projects, onto the table 13 or the patient Pa, a light beam indicating the irradiation area adjusted by the collimator 11b in the irradiation direction adjusted by the collimator 11b. In the collimator lamp 11c, a length of the irradiation area in one direction is indicated by a linear light extending in the one direction. In the collimator lamp 11c, a length of the irradiation area in another direction perpendicular to the one direction is indicated by a linear light extending in the another direction. In other words, the collimator lamp 11c indicates the irradiation area by using cross-shaped light beams. Also, the collimator lamp 11c indicates a center position Ce of the collimator lamp 11c by using an intersection of the cross-shaped light beams (see FIG. 11). Here, the irradiation direction refers to a direction from the X-ray irradiator 11 toward the detector 15.

The grip 11d is a handle that is gripped by the radiologic technologist Ra when the X-ray irradiator 11 is moved. Specifically, the radiologic technologist Ra grips the grip 11d when moving the X-ray irradiator 11 in the X direction, the Y direction and the Z direction, or rotating the X-ray irradiator 11 about the axis extending in the Z direction and about the plurality of predetermined rotation axes extending in directions perpendicular to the Z direction. In this way, the radiologic technologist Ra integrally moves the X-ray irradiator 11 by linearly moving or rotating the X-ray irradiator in desired directions while gripping the grip 11d. Here, the radiologic technologist Ra may manually move the X-ray irradiator 11 by touching a part of the X-ray irradiator 11 other than the grip 11d.

### <Irradiator-Moving Mechanism>

As shown in FIG. 2, the irradiator-moving mechanism 12 is configured to change a relative position of the X-ray irradiator 11 relative to the patient Pa by manually moving the X-ray irradiator 11 by the radiologic technologist Ra.

Specifically, the irradiator-moving mechanism 12 includes an X-direction movement rail 12a, a Y-direction movement rail 12b, and a telescopic mechanism 12c. The X-direction movement rail 12a is fixed to a ceiling. The Y-direction movement rail 12b is attached to the X-direction movement rail 12a movably in the X direction. The telescopic mechanism 12c is attached to the Y-direction movement rail 12b to be able to telescope in the Z directions. The X-ray irradiator 11 is attached to an end of the Z2 direction of the telescopic mechanism 12c to be rotable about a plurality of predetermined rotation axes extending in directions perpendicular to the Z direction. Accordingly, in the X-ray imaging apparatus 1, X-ray images Gx (see FIG. 5) are captured through X-rays with which the patient is irradiated from the X-ray irradiator 11 suspended from the ceiling through the irradiator-moving mechanism 12.

The X-direction movement rail 12a is configured to guide movement of the X-ray irradiator 11 in the X1 direction or the X2 direction when the radiologic technologist Ra manually moves the X-ray irradiator 11. The X-direction movement rail 12a is a guide rail extending in the X direction. The Y-direction movement rail 12b is configured to guide movement of the X-ray irradiator 11 in the Y1 direction or the Y2 direction when the radiologic technologist Ra manually moves the X-ray irradiator 11. The Y-direction movement rail 12b is a guide rail extending in the Y direction. The telescopic mechanism 12c is configured to extend in the Z1 direction or retract in the Z2 direction when the radiologic technologist Ra manually moves the X-ray irradiator 11.

### <Table>

The table 13 is a plate-shaped part having a placement surface on which the patient Pa is placed. The table 13 is attached to an end of the table-moving mechanism 14 on the Z1-direction side. The table 13 can be moved in the X, Y, and Z directions relative to the X-ray irradiator 11 by the table-moving mechanism 14. In other words, the radiologic technologist Ra integrally moves the table 13 by linearly moving or rotating the table in desired directions while gripping the grip 11d. Here, the radiologic technologist Ra may manually move the table 13 by touching a part of the table 13 other than the grip.

### <Table-Moving Mechanism>

The table-moving mechanism 14 is configured to change a relative position of patient Pa on the table 13 relative to the X-ray irradiator 11 by manually moving the table 13 by the radiologic technologist Ra.

Specifically, the table-moving mechanism 14 includes an X-direction movement rail 14a, a Y-direction movement rail 14b, and a lifting mechanism 14c. The X-direction movement rail 14a is fixed to a floor of an imaging room Rm. The Y-direction movement rail 14b is attached to the X-direction movement rail 14a movably in the X direction. The lifting mechanism 14c is attached to the Y-direction movement rail 14b and can move in the Y-direction. The table 13 is attached to the lifting mechanism 14c movably in the Z direction. Accordingly, the table 13 can be moved in the X, Y, and Z directions.

The X-direction movement rail 14a is configured to guide movement of the table 13 in the X1 direction or the X2 direction when the radiologic technologist Ra manually moves the table 13. The Y-direction movement rail 14b is configured to guide movement of the table 13 in the Y1 direction or the Y2 direction when the radiologic technologist Ra manually moves the table 13. The lifting mechanism 14c is configured to guide upward movement of the table 13 in the Z1 direction or downward movement of the table 13 in the Z2 direction when when the radiologic technologist Ra manually moves the table 13.

### <Detector>

The detector 15 is configured to detect X-rays with which the patient Pa is irradiated from the X-ray irradiator 11 and that pass through the patient Pa. The detector 15 is an FPD (Flat Panel Detector). The detector 15 is used when images are captured with the patient being lying (decubitus or lateral position) on the table 13. The detector 15 is accommodated in an accommodation part provided in the table 13 on the Z2-direction side to be movable in and out. The accommodation part is attached to the detector-moving mechanism 16. The accommodation part is configured to be movable in the X and Y directions relative to the table 13 by the detector-moving mechanism 16.

### <Detector-Moving Mechanism>

The detector-moving mechanism 16 is configured to move the detector 15, which is held in the accommodation part, in the X and Y directions by allowing the radiologic technologist Ra to manually move the accommodation part in the X and Y directions. Here, the detector-moving mechanism 16 may be a mechanism that moves the accommodation part of the outer edge only in the X direction by allowing the radiologic technologist Ra to manually move the accommodation part. Also, the radiologic technologist Ra integrally moves the detector in a desired direction by linearly moving the detector 15 while touching the accommodation part.

Specifically, the detector-moving mechanism 16 includes an X-direction movement rail 16a and a Y-direction movement rail 16b. The X-direction movement rail 16a is fixed to the table 13. The Y-direction movement rail 16b is attached to the X-direction movement rail 16a movably in the X direction. The accommodation part is attached to the Y-direction movement rail 16b movably in the Y direction.

The X-direction movement rail 16a is configured to guide movement of the accommodation part in the X1 direction or the X2 direction when the radiologic technologist Ra manually moves the accommodation part. The Y-direction movement rail 16b is configured to guide movement of the accommodation part in the Y1 direction or the Y2 direction when the radiologic technologist Ra manually moves the accommodation part.

### <Communicator>

The communicator 17 is a device for communicating with an image processing apparatus 2 through wired or wireless connection. The communicator 17 is configured to receive signals from the image processing apparatus 2 that instruct the start of capturing X-ray images Gx of the patient Pa using the X-ray imaging apparatus 1. The communicator 17 is configured to transmit detection signals from the detector 15 to the image processing apparatus 2 based on signals from the image processing apparatus 2.

### <Controller>

The apparatus controller 18 is configured to entirely control the X-ray imaging apparatus 1. The controller 18 includes a CPU (Central Processing Unit), and the like.

### (Image Processing Apparatus)

The image processing apparatus 2 is configured to perform processing to generate X-ray images Gx of the patient Pa based on detection signals received from the detector 15 of the X-ray imaging apparatus 1. The image processing apparatus 2 includes a controller 21, a storage 22, a display 23, and a communicator 24. Here, the display 23 is an example of a “display apparatus” in the claims.

The controller 21 includes a CPU, a GPU (Graphics Processing Unit) and the like. The storage 22 includes a non-volatile storage medium such as HDD (Hard Disk Drive) or SSD (Solid State Drive). The display 23 is configured to display an X-ray image Gx generated by the controller 21. The display 23 includes a liquid crystal display, an organic EL display, or the like. The communicator 24 is a device for communicating with the X-ray imaging apparatus 1 and the image server 3 through wired or wireless connection. The communicator 24 is configured to transmit signals that instruct the start of capturing X-ray images Gx of the patient Pa using the X-ray imaging apparatus 1 to the X-ray imaging apparatus 1. Here, the signals that gives instructions to start capturing X-ray images Gx of the patient Pa using the X-ray imaging apparatus 1 are signals that are generated in response to an operation from the radiologic technologist Ra that gives instructions to start capturing the images. The communicator 24 is configured to receive detection signals from the detector 15. The communicator 24 is configured to transmit X-ray images Gx to the image server 3.

### (Image Server)

As shown in FIG. 1, the image server 3 is configured to receive X-ray images Gx based on instructions from the image processing apparatus 2 and to save the X-ray images into its storage. The image server 3 is configured to transmit the X-ray images Gx stored in the storage to the mobile terminal 4 based on instructions from the mobile terminal 4.

This type of image server 3 serves as a so-called PACS (Picture Archiving and Communication Systems). The X-ray images Gx are stored in a format compliant with DICOM (Digital Imaging and Communication in Medicine) in the image server 3.

Specifically, the image server 3 includes a controller (not shown), the storage (not shown), and a communicator (not shown). The controller includes a CPU. The storage includes a non-volatile storage medium such as HDD or SSD. The communicator is a device for communicating with the X-ray imaging apparatus 1 and the image processing apparatus 2 through wired or wireless connection. The communicator is configured to receive X-ray images Gx from the image processing apparatus 2. The communicator is configured to transmit the X-ray image Gx stored in the storage to the mobile terminal 4.

### (Mobile Terminal)

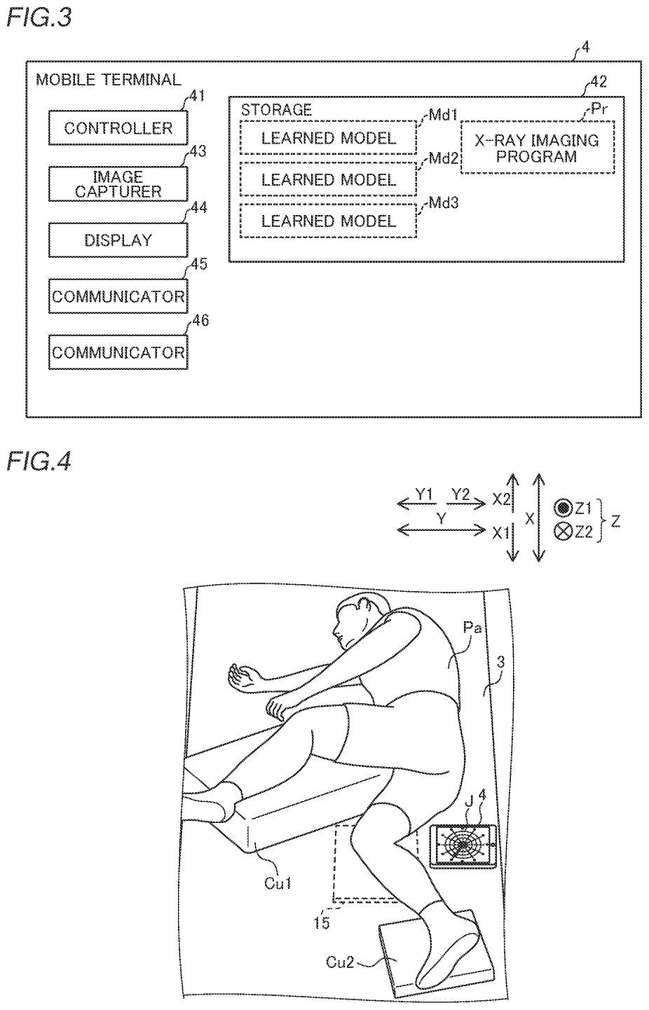

As shown in FIG. 1 and FIG. 3, the mobile terminal 4 has a function of assisting the radiologic technologist Ra in capturing X-ray images Gx of the patient Pa. The mobile terminal 4 is constructed of a tablet device. The mobile terminal 4 is carried by the radiologic technologist Ra. The mobile terminal 4 is provided separately from the X-ray imaging apparatus 1, the image processing apparatus 2, and the image server 3.

Specifically, the mobile terminal 4 includes a controller 41, a storage 42, an image capturer 43, a display 44, a communicator 45, and a communicator 46.

The controller 41 includes a CPU, and the like. The storage 42 includes a volatile storage medium such as flash memory. The storage 42 stores a learned model Md1, a learned model Md2, and a learned model Md3. Also, the storage 42 stores an X-ray imaging program Pr. Also, the learned model Md1, the learned model Md2, the learned model Md3 and the X-ray imaging program Pr will be described in detail later.

The image capturer 43 is a camera. The display 44 is an organic EL display, or the like. The communicator 45 is a device for communicating with an external server, the image servers 3 and the like through wired or wireless communication. The communicator 46 is a device for communicating with a portable storage medium such as USB memory.

### (Learned Model)

The learned model Md1, the learned model Md2, the learned model Md3 are learned models that have been produced by machine learning using X-ray images Gx of imaging target parts as input data. Here, the imaging target parts include joint parts such as shoulders and elbows in artificial bones or bones, for example. Also, the learned model Md1, the learned model Md2, the learned model Md3 are models acquired based on machine learning such as U-Net, for example. Here, the machine learning using X-ray images Gx as input data can be any of supervised learning, unsupervised learning, and reinforcement learning.

The learned model Md1 and the learned model Md2 are exemplary learned models that are to be used when capturing images of a medial condyle of a femur (see FIG. 5) and a lateral condyle of the femur (see FIG. 5) in a knee joint of the patient Pa. The learned model Md3 is a learned model that is to be used when capturing an elbow joint of patient Pa, for example.

### (X-Ray Imaging Program)

As shown in FIGS. 4 to 6, the mobile terminal 4 is configured to perform control, when capturing X-ray images Gx, to display imaging assistance information J so as to assist in capturing the images based on the X-ray imaging program Pr. The imaging assistance information J is information that suggests, to the radiologic technologist Ra, a change and the like when capturing a subsequent X-ray image Gx (main captured X-ray image Gx2) to make the subsequent X-ray image Gx more appropriate for diagnosis than an initial X-ray image Gx (pre-captured X-ray image Gx1) if capturing a plurality of X-ray images Gx. The following description describes an exemplary case in which images of a medial condyle and a lateral condyle of the femur of the patient Pa as a situation in which the imaging assistance information J is required. Here, the imaging assistance information J may be used when capturing other X-ray images Gx, and the situation in which the imaging assistance information J is required should not be limited to specific interpretations.

As shown in FIGS. 4 and 5, the degree of difficulty in capturing X-ray images Gx of a side of the knee joint to be used for diagnosis of diseases around the knee joint disorders such as osteochondritis dissecans and osteoarthritis is high even if the radiologic technologists Ra is experienced.

Specifically, in order to accurately diagnose a disease, it is necessary to capture an X-ray image Gx in which an outer edge Bi of the medial condyle of the femur (indicated by a thick solid line in FIG. 5) overlaps an outer edge Bo of the lateral condyle of the femur (indicated by a thick dashed line in FIG. 5). Here, the outer edge Bi of the medial condyle of the femur is indicated by the thick solid line in FIG. 5, while the outer edge Bo of the lateral condyle of the femur is indicated by the thick dotted line in FIG. 5.

in this image capturing, the radiologic technologist Ra adjusts a rotation degree of a thigh and a lower leg, and an elevation degree of the lower leg (bending angle of the knee) by using a cushion Cu1 and a cushion Cu2 to properly position the knee of the patient Pa lying in a decubitus position on a top surface of the table 13. Subsequently, the radiologic technologist Ra captures an X-ray image Gx (pre-captured X-ray image Gx1) of the knee of the patient Pa lying in the decubitus position after the adjustment.

However, since individual differences exist in shapes of bones and distributions of flesh on legs, even if the radiologic technologist Ra is experienced, it is very difficult to adjust an imaging position based on the appearance of the patient Pa so as to overlap the outer edge Bi of the medial condyle of the femur with the outer edge Bo of the lateral condyle of the femur when capturing the X-ray image Gx.

To address this, in order to capture an X-ray image Gx in which the outer edge Bi of the medial condyle of the femur overlaps the outer edge Bo of the lateral condyle of the femur to allow the radiologic technologist to accurately diagnose the disease, it is necessary to correct the imaging position of the X-ray irradiator 11 based on the pre-captured X-ray image Gx1, which has been captured. Subsequently, the radiologic technologist Ra captures an X-ray image Gx of the knee of the patient Pa lying in the decubitus position after correcting the imaging position of the X-ray irradiator 11. To capture an X-ray image Gx that allows the radiologic technologist to accurately diagnose the disease, it is necessary to repeatedly capture the pre-captured X-ray image Gx1 and then correct the imaging position. In this case, the exposure dose to the patient Pa and the imaging time will increase.

For this reason, the imaging assistance information J is required to prevent the increase of the exposure dose to the patient Pa and imaging time, but it is conceivable that the X-ray imaging apparatus 1 and the image processing apparatus 2 are not manufactured for the common purpose of displaying the imaging assistance information J. In such a case, even if the specifications of the controller 18 of the X-ray imaging apparatus 1 meet specifications for displaying the imaging assistance information J, since the specifications of the controller 21 of the image processing apparatus 2 do not meet the specifications for displaying the imaging assistance information J, it is conceivable that installation of a program for displaying the imaging assistance information J on the image processing apparatus 2 is difficult.

To address this, as shown in FIG. 4, in the first embodiment, imaging assistance information J for assistance of capture of the main captured X-ray image Gx2 is displayed on a general-purpose mobile terminal 4. Since the general-purpose mobile terminal 4 includes not only the controller 41 but also the display 44, the mobile terminal 4 can both acquire and display the imaging assistance information J.

As shown in FIGS. 5 to 7, the controller 41 of the mobile terminal 4 is configured to perform control to acquire the imaging assistance information J for assistance in capturing the main captured X-ray image Gx2, which is captured through X-rays with which the patient Pa is irradiated from the X-ray irradiator 11, based on an X-ray image Gx (pre-captured X-ray image Gx1) that is captured through X-rays, which is captured through X-rays with which the patient Pa is irradiated from the X-ray irradiator 11 and that pass through the patient, after capturing the pre-captured X-ray image Gx1. Also, the controller 41 is configured to perform control to display the imaging assistance information J acquired in the controller 41 on the display 44.

Specifically, as shown in FIG. 5, the controller 41 is configured to perform control to acquire the pre-captured X-ray image Gx1, which is captured through X-rays with which the patient Pa is irradiated from the X-ray irradiator 11 and that pass through the patient. Here, the controller 41 is configured to control to acquire the pre-captured X-ray image Gx1 from the image server 3 via the network by using the communicator 45. The pre-capture X-ray image Gx1 is an image that is captured under an imaging condition in which a dose of X-rays when the pre-captured X-ray image is captured is smaller than a dose of X-rays when the main captured X-ray image Gx2 is captured. The imaging condition in which the dose of X-rays when the pre-captured X-ray image is captured is smaller is stored in the image processing apparatus 2 by the radiologic technologist Ra in advance. Also, the pre-captured X-ray image Gx1 is an image that is captured at the time after patient Pa has entered the imaging room Rm and before capturing the main captured X-ray image Gx2. As described above, the pre-captured X-ray image Gx1 is an image that is captured after the radiologic technologist Ra adjusts the rotation degree of the thigh and the lower leg, and the elevation degree of the lower leg (bending angle of the knee) by using the cushion Cu1 and the cushion Cu2 to properly position the knee of the patient Pa lying in the decubitus position on the top surface of the table 13.

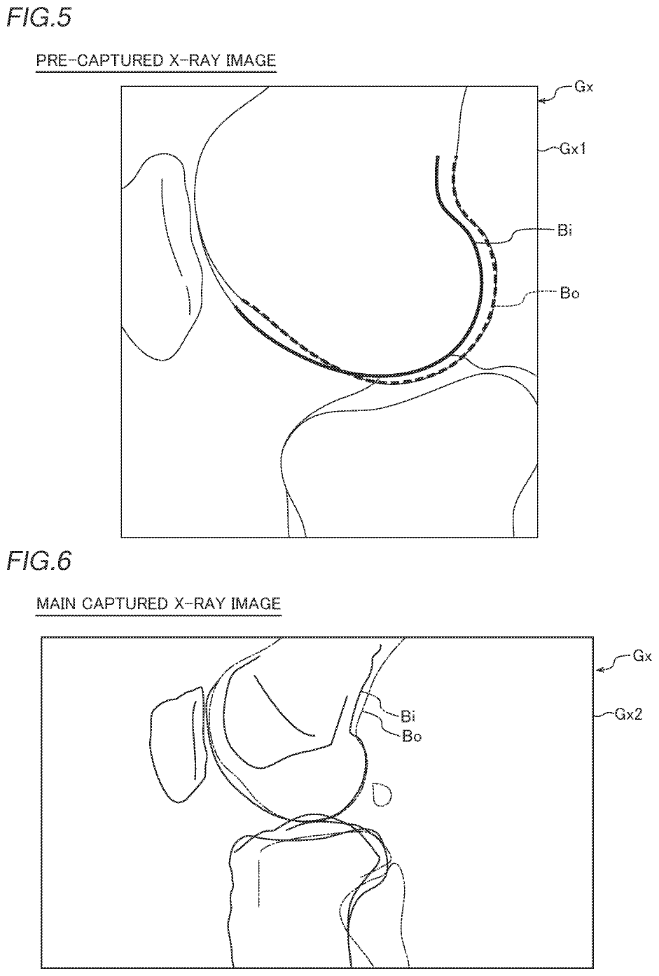

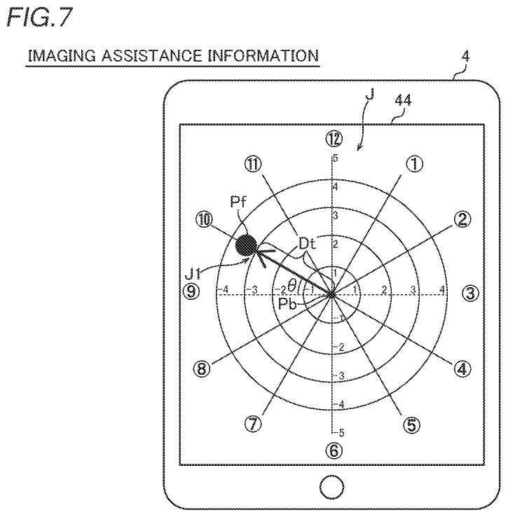

Also, as shown in FIGS. 6 and 7, the controller 41 is configured to perform control to acquire the imaging assistance information J for assistance in capturing the main captured X-ray image Gx2, which is captured through X-rays with which the patient Pa is irradiated from the X-ray irradiator 11, based on the pre-captured X-ray image Gx1 after capturing the pre-captured X-ray image Gx1. Here, the controller 41 is configured to perform control to acquire position correction information J1 for the radiologic technologist Ra to manually correct a relative position between the X-ray irradiator 11 and the patient Pa based on the pre-captured X-ray image Gx1 as the imaging assistance information J. Accordingly, the imaging assistance information J includes the position correction information J1.

Here, the position correction information J1 includes information on a movement direction θ in horizontal directions and a movement distance Dt when the radiologic technologist Ra manually moves the X-ray irradiator 11 and the patient Pa relative to each other. In addition, it is assumed that the radiologic technologist Ra moves the X-ray irradiator 11 and the patient Pa relative to each other by manually moving the X-ray irradiator 11.

As shown in FIG. 7, the position correction information J1 is indicated on the display 44. The position correction information J1 is indicated by an arrow and a circular mark on the display 44. A Pb point, which is a base end of the arrow indicated on the display 44, is information on a pre-correction position for positioning a current position of the X-ray irradiator 11. Also, a Pf point, which is a center position of the circular mark in contact with a head end of the arrow indicated on the display 44, is information on a corrected position of the X-ray irradiator 11 to be moved from the pre-correction position for positional correction. Here, the current position of the X-ray irradiator 11 is a spatial position of the X-ray irradiator 11 when the pre-captured X-ray image Gx1 is captured after adjustment by the radiologic technologist Ra. The current position of the X-ray irradiator 11 is a reference position in the positional correction. For this reason, it is necessary to fix the X-ray irradiator 11 until the start of movement of the X-ray irradiator 11 from the pre-correction position to the corrected position after the pre-captured X-ray image Gx1 is captured.

Also, the display 44 indicates a radar chart that includes a plurality of concentric circles having different diameters for allowing the radiologic technologist Ra to recognize distances, a plurality of linear lines extending in radial directions of the concentric circles for allowing the radiologic technologist Ra to recognize directions, and numbers assigned to the plurality of linear lines. The plurality of lines are twelve lines indicated on the display 44. The plurality of linear lines are spaced at predetermined constant angles (approximately 30 degrees in FIG. 7). The numbers assigned to the plurality of linear lines are numbered in order from 1 to 12 clockwise. Here, the mobile terminal 4 is arranged on the table 13 with the X1 direction agreeing with the linear line that extends toward the number 3. In this case, the mobile terminal 4 is arranged on the table 13 with the X2 direction agreeing with the linear line that extends toward the number 9, the Y1 direction agreeing with the linear line that extends toward the number 6, and the Y2 direction agreeing with the linear line that extends toward the number 12.

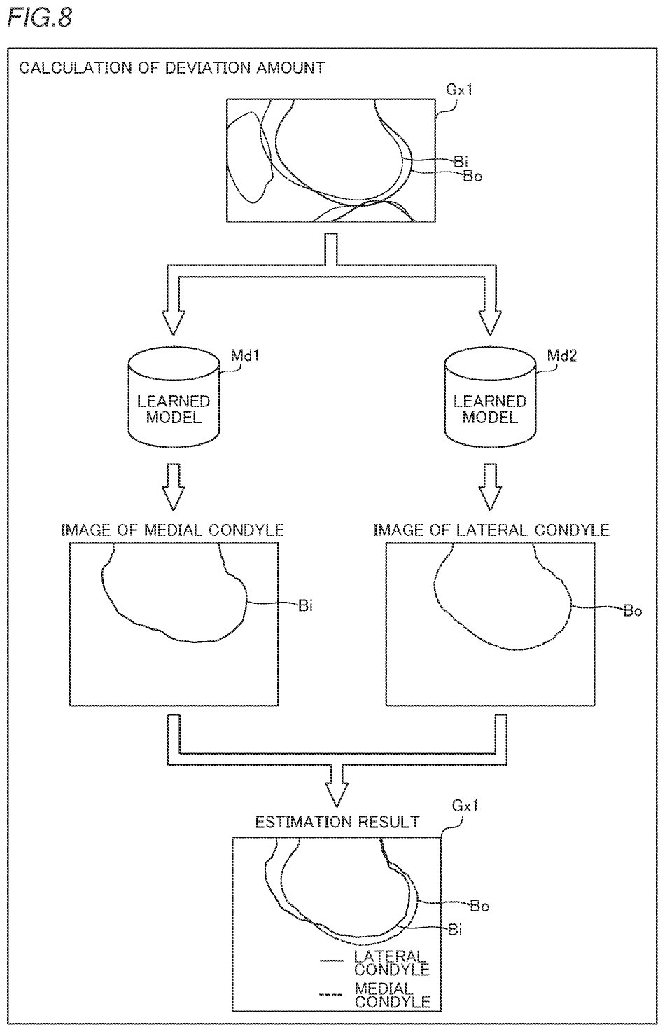

As illustratively shown in FIG. 8, the position correction information J1 is information for correcting a positional deviation between the outer edge Bi of the medial condyle and the outer edge Bo of the lateral condyle included in the bone of the patient Pa in the pre-captured X-ray image Gx1 in a direction perpendicular to the irradiation direction of the X-ray irradiator 11 based on the pre-captured X-ray image Gx1. Here, in exemplary arrangement, the direction perpendicular to the irradiation direction of the X-ray irradiator 11 is a horizontal direction.

Specifically, the controller 41 is configured to perform control to find, based on the pre-capture X-ray image Gx1, the outer edge Bi of the medial condyle, which is a part of the outer edge on the X-ray irradiator 11 side in the irradiation direction included in the bone of the patient Pa in the pre-capture X-ray image Gx1, and the outer edge Bo of the lateral condyle, which is a part of the outer edge on a side opposite to the X-ray irradiator 11 side included in the bone of the patient, to acquire the position correction information J1.

That is, as shown in FIGS. 8 and 9, the controller 41 is configured to perform control to find the outer edge Bi of the medial condyle based on analysis performed on the pre-captured X-ray image Gx1 acquired through the learned model Md1. The controller 41 is configured to perform control to find the outer edge Bo of the lateral condyle based on analysis through the learned model Md2 acquired. The controller 41 is configured to perform control to display the pre-captured X-ray image Gx1 on the display 44 of the mobile terminal 4 with identification marks Ma being indicated to identify the outer edge Bi found of the medial condyle, which is a part of the outer edge on the X-ray irradiator 11 side, and the outer edge Bo found of the lateral condyle, which is a part of the outer edge on the side opposite to the X-ray irradiator 11 side. Here, in FIG. 9, the identification marks Ma are a thick solid line for emphasizing the outer edge Bi of the medial condyle and a thick dotted line for emphasizing the outer edge Bo of the lateral condyle. Here, the identification marks Ma may be any forms, such as different color lines, as long as they emphasize the outer edge Bo of the lateral condyle and the outer edge Bi of the medial condyle to be easily identified.

The controller 41 is configured to perform control to receive determination by the radiologic technologist Ra on whether to perform position correction to align the outer edge Bi of the medial condyle, which is a part of the outer edge on the X-ray irradiator 11 side, and the outer edge Bo of the lateral condyle, which is a part of the outer edge on the side opposite to the X-ray irradiator 11 side, with each other. That is, if determining to perform the position correction, the radiologic technologist Ra presses an indication part “Yes” in a box “start acquisition of position correction information J1” in FIG. 9. In this case, the controller 41 is configured to start control to acquire the position correction information J1. Also, if determining not to perform the position correction, the radiologic technologist Ra presses an indication part “No” in the box “start acquisition of position correction information J1” in FIG. 9. In this case, the controller 41 is configured not to start the control to acquire the position correction information J1.

The Controller 41 is configured to perform control to calculate deviation (a deviation amount and a deviation direction) between the outer edge Bi of the medial condyle and the outer edge Bo of the lateral condyle based on the outer edge Bi of the medial condyle and the outer edge Bo of the lateral condyle that are found (estimated) from the pre-captured X-ray image Gx1 after starting the control to acquire the position correction information J1.

Specifically, the controller 41 is configured to control to search for the positions of the outer edge Bi of the medial condyle and the outer edge Bo of the lateral condyle that bring the medial condyle and the lateral condyle that are found to substantially agree with each other by moving one of the medial condyle and the lateral condyle. Here, the positions to be searched are positions that minimize an area formed by the deviation between the outer edge Bi of the medial condyle and the outer edge Bo of the lateral condyle after the moving. Accordingly, the deviation between the outer edge Bi of the medial condyle and the outer edge Bo of the lateral condyle is found by using the deviation amount and the deviation direction.

The controller 41 is configured to perform control to calculate, based on the deviation amount and the deviation direction found, the position correction information J1 for correcting the relative position of the X-ray irradiator 11 relative to the knee of the patient Pa to a position at which the main captured X-ray image Gx2 (see FIG. 6) can be captured with the outer edge Bi of the medial condyle and the outer edge Bo of the lateral condyle overlapping each other when capturing an image of the knee of the patient Pa. That is, the controller 41 is configured to perform control to calculate the movement direction θ and the movement distance Dt of the X-ray irradiator 11 as the position correction information J1 based on the deviation amount and the deviation direction.

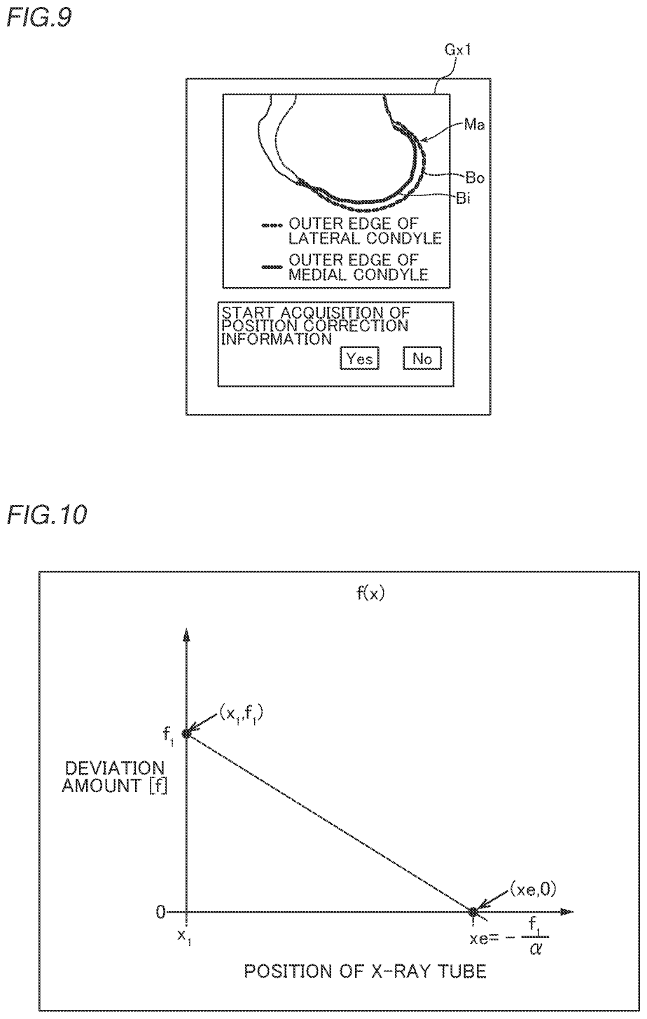

Specifically, as shown in FIG. 10, the controller 41 is configured to perform control to calculate the movement direction θ and the movement distance Dt as the position correction information J1 based on a linear function f(x) that represents a linear change in deviation amount f between the outer edge Bi of the medial condyle and the outer edge Bo of the lateral condyle changing with an X-ray irradiation point of the X-ray irradiator 11 (X-ray tube position x). Here, the linear function f(x) is calculated based on a deviation amount f1 and a slope a. Here, the slope a is acquired based on various types of parameters including parameters of the apparatus, such as SID (Source to image receptor distance), and parameters set in accordance with the patient Pa when capturing images of the patient. This linear function f(x) is used to estimate an estimated position xe as the position at which the main captured X-ray image Gx2 can be captured with the outer edge Bi of the medial condyle and the outer edge Bo of the lateral condyle overlapping each other.

Accordingly, the controller 41 is configured to perform control to calculate the movement direction θ and the movement amount of the X-ray irradiator 11 as the position correction information J1 based on comparison between the estimated position xe and the current position of the X-ray irradiator 11. Here, the estimated position xe is information on the aforementioned corrected position of the X-ray irradiator 11. In addition, the mobile terminal 4 acquires the current position of the X-ray irradiator 11 based on input of numerical values measured by the radiologic technologist Ra using a scale into the mobile terminal 4. Consequently, the position correction information J1 is acquired.

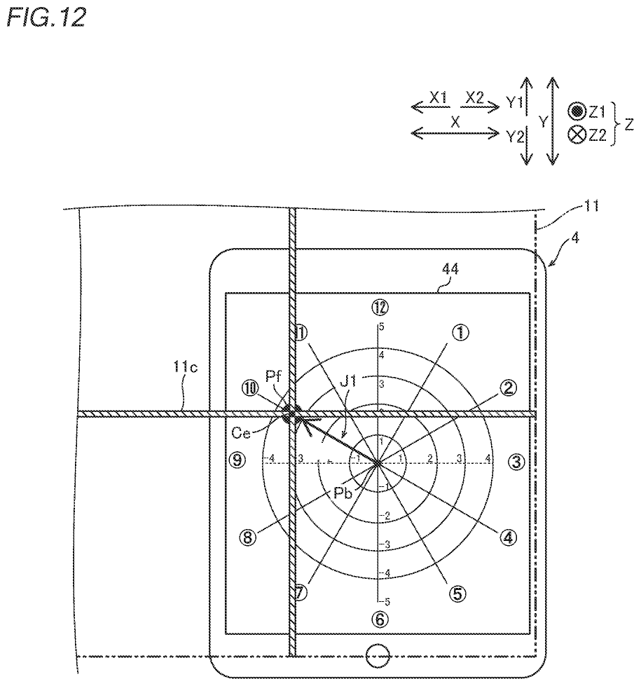

As shown in FIGS. 11 and 12, the controller 41 is configured to control to display the imaging assistance information J on the display 44 of the mobile terminal 4 before capturing the main captured X-ray image Gx2. Here, as shown in FIG. 11, the radiologic technologist Ra positions the base end of the arrow, which is the position correction information J1, at the center position Ce, which is the intersection of the cross-shaped light beams emitted for irradiation from the collimator lamp 11c, by moving the mobile terminal 4. At this time, as described above, the spatial position of the X-ray irradiator 11 is maintained as the position at which the pre-captured X-ray image Gx1 has been captured. Subsequently, as shown in FIG. 12, the radiologic technologist Ra positions the center position Ce, which is the intersection of the cross-shaped light beams emitted for irradiation from the collimator lamp 11c, to the Pf point, which is the position correction information J1 and is a center position of the circular mark in contact with the head end of the arrow, by moving the X-ray irradiator 11 while grasping the grip 11d.

Accordingly, the deviation between the outer edge Bi of the medial condyle and the outer edge Bo of the lateral condyle can be corrected by moving the X-ray irradiator 11 in a horizontal direction to change its horizontal position without changing a rotation angle of the X-ray irradiator 11 by fan beam characteristics of the X-rays emitted for irradiation from the X-ray irradiator 11. That is, since the irradiation direction of X-rays passing through the outer edge Bi of the medial condyle and the outer edge Bo of the lateral condyle is changed by moving the X-ray irradiator 11 in a horizontal direction to change its position in a horizontal direction, it is possible to correct the deviation between the outer edge Bi of the medial condyle and the outer edge Bo of the lateral condyle without changing the rotation angle of the X-ray irradiator 11.

The radiologic technologist Ra moves to a console room in which the image processing apparatus 2 is placed and is outside the imaging room Rm, after changing position of the X-ray irradiator 11 in the horizontal direction while holding the mobile terminal 4. Subsequently, the radiologic technologist Ra conducts an operation to start the X-ray irradiation using the X-ray imaging apparatus 1. That is, the X-ray imaging apparatus 1 is configured to capture, based on the reception of the operation from the radiologic technologist Ra, the main captured X-ray image Gx2 under an imaging condition in which the dose of X-rays when the main captured X-ray image is captured is higher than the dose of X-rays when the pre-captured X-ray image Gx1 is captured after the pre-captured X-ray image Gx1 is captured. The imaging condition in which the dose of X-rays when the pre-captured X-ray image is captured is higher is stored in the image processing apparatus 2 by the radiologic technologist Ra in advance. Consequently, the main captured X-ray image Gx2 is acquired. The main captured X-ray image Gx2 captured is transmitted from the image processing apparatus 2 to the image server 3 and stored in the image server.

### (X-Ray Imaging Method)

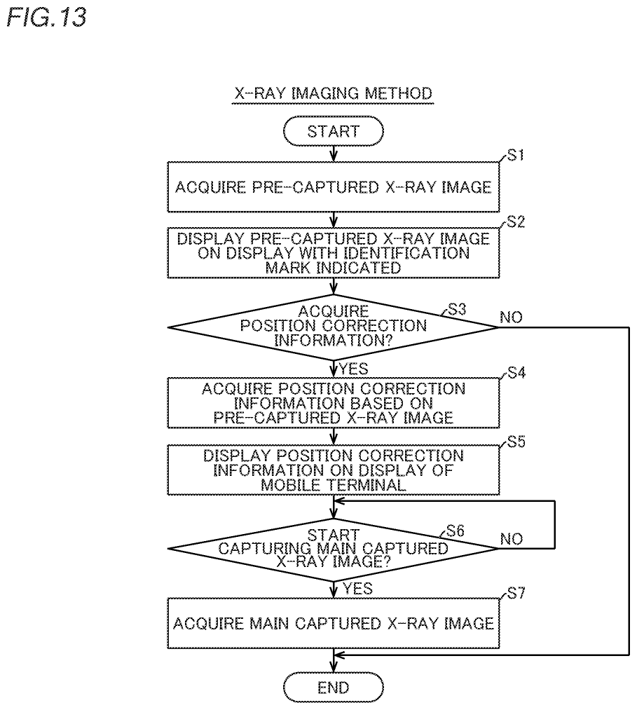

The following description describes an X-ray imaging method performed in the X-ray imaging system 100 with reference to FIG. 13.

As shown in FIG. 13, the mobile terminal 4 acquires the pre-captured X-ray image Gx1, which is captured through X-rays with which the patient Pa is irradiated from the X-ray irradiator 11 and that pass through the patient, in step S1. Specifically, the X-ray imaging apparatus 1 captures the pre-captured X-ray image Gx1 under an imaging condition in which a dose of X-rays when the pre-captured X-ray image is captured is smaller than a dose of X-rays when the main captured X-ray image Gx2 is captured. The imaging condition in which the dose of X-rays when the pre-captured X-ray image is captured is smaller is set by the radiologic technologist Ra in advance. The image processing apparatus 2 generates the pre-captured X-ray image Gx1 of the patient Pa based on detection signals captured by the X-ray imaging apparatus 1. The image processing apparatus 2 transmits the pre-captured X-ray image Gx1 to the image server 3. Accordingly, the mobile terminal 4 acquires the pre-captured X-ray image Gx1 from the image server 3 via the network.

In step S2, the mobile terminal 4 displays the pre-captured X-ray image Gx1 on the display 44 with the identification mark Ma being indicated. Specifically, the mobile terminal 4 finds, based on the pre-capture X-ray image Gx1, a part of the outer edge on the X-ray irradiator 11 side (e.g., the outer edge Bi of the medial condyle) in the irradiation direction included in the bone of the patient Pa in the pre-capture X-ray image Gx1, and a part of the outer edge on a side opposite to the X-ray irradiator 11 side (e.g., the outer edge Bo of the lateral condyle) to acquire the position correction information J1. The mobile terminal 4 displays the pre-captured X-ray image Gx1 on the display 44 of the mobile terminal 4 with the identification mark Ma being indicated to find the part of the outer edge on the X-ray irradiator side and the part of the outer edge on the side opposite to the X-ray irradiator side.

In step S3, the mobile terminal 4 determines whether to acquire the position correction information J1. If the position correction information J1 is not acquired, the X-ray imaging method is terminated, and if the position correction information J1 is acquired, the procedure goes to step S4. Specifically, the mobile terminal 4 receives determination by the radiologic technologist Ra on whether to perform position correction to align the part of the outer edge on the X-ray irradiator 11 side (e.g., the outer edge Bi of the medial condyle) with the part of the outer edge on the side opposite to the X-ray irradiator 11 side (e.g., the outer edge Bo of the lateral condyle). If the operation from the radiologic technologist Ra that indicates not to acquire the position correction information J1 is received, the X-ray imaging method is terminated, and if the operation from the radiologic technologist Ra that indicates to acquire the position correction information J1 is received, the procedure goes to step S4.

In step S4, the mobile terminal 4 acquires the position correction information J1 based on the pre-captured X-ray image Gx1. That is, the mobile terminal 4 acquires the position correction information J1 as the imaging assistance information J for assistance in capturing the main captured X-ray image Gx2 captured through X-rays with which the patient is irradiated from the X-ray irradiator 11 based on the pre-captured X-ray image Gx1 after capturing the pre-captured X-ray image Gx1.

In step S5, the mobile terminal 4 displays the position correction information J1 on the display 44 based on the acquisition of the position correction information J1. Specifically, the mobile terminal 4 displays an arrow on the display 44 as the position correction information J1, which includes information on the movement direction θ in horizontal directions and the movement distance Dt when the radiologic technologist Ra manually moves the X-ray irradiator 11 and the patient Pa relative to each other. Here, the base end of the arrow (see FIG. 11) is information on the pre-correction position for positioning the current position of the X-ray irradiator 11. The circular mark (see FIG. 12) in contact with the head end of the arrow is information on a corrected position of the X-ray irradiator 11 to be moved from the pre-correction position for positional correction. Accordingly, the mobile terminal 4 indicates the position correction information J1 as the imaging assistance information J on the display 44 of the mobile terminal 4 before the main captured X-ray image Gx2 is captured.

Here, the radiologic technologist Ra places the mobile terminal on the table 13 with the base end of the arrow, which is the position correction information J1, being aligned with the center position Ce, which is the intersection of the cross-shaped light beams emitted for irradiation from the collimator lamp 11c, by moving the mobile terminal 4 after step S5. In addition, the numbers indicated on the display 44 of the mobile terminal 4 are used so that the orientation of the mobile terminal 4 on the table 13 is previously specified, while the number 3 is orientated on the table 13 toward the head side of the patient Pa, to bring a linear line that extends from the number 3 to the number 9 to agree with a longitudinal direction of the table 13, for example. Subsequently, the radiologic technologist Ra positions the center position Ce, which is the intersection of the cross-shaped light beams emitted for irradiation from the collimator lamp 11c, to the circular mark, which is the position correction information J1 and is in contact with the head end of the arrow, by moving the X-ray irradiator 11 while grasping the grip 11d. The radiologic technologist Ra moves to the console room outside of the imaging room Rm, after changing the position of the X-ray irradiator 11 in the horizontal direction while holding the mobile terminal 4. Subsequently, the radiologic technologist Ra conducts an operation to start the X-ray irradiation using the X-ray imaging apparatus 1.

In step S6, the X-ray imaging apparatus 1 determines whether the operation from the radiologic technologist Ra to start capturing the main captured X-ray image Gx2 is received. If the operation from the radiologic technologist Ra to start capturing the main captured X-ray image Gx2 is received, the procedure goes to step S7, and if the operation from the radiologic technologist Ra to start capturing the main captured X-ray image Gx2 is not received, step S6 is repeated.

In step S7, the image processing apparatus 2 acquires the main captured X-ray image Gx2. Specifically, the X-ray imaging apparatus 1 irradiates the patient Pa with X-rays toward the patient based on the reception of the operation from the radiologic technologist Ra to start capturing the main captured X-ray image Gx2. Here, the X-ray imaging apparatus 1 is configured to capture, based on the reception of the operation by the radiologic technologist Ra, the main captured X-ray image Gx2 under an imaging condition in which the dose of X-rays when the main captured X-ray image is captured is higher than the dose of X-rays when the pre-captured X-ray image Gx1 is captured after the pre-captured X-ray image Gx1 is captured. The imaging condition in which the dose of X-rays when the pre-captured X-ray image is captured is higher is specified by the radiologic technologist Ra in advance. Subsequently, the image processing apparatus 2 generates the main captured X-ray image Gx2 based on detection signals detected by the detector 15.

After step S7, the X-ray imaging method is terminated.

### Advantages of First Embodiment

In the first embodiment, the following advantages are obtained.

In the first embodiment, as described above, the X-ray imaging method includes step S1 of acquiring, in the mobile terminal 4, imaging assistance information J for assistance in capturing the main captured X-ray image Gx2 captured through X-rays with which the patient is irradiated from the X-ray irradiator 11 based on the pre-captured X-ray image Gx1 after capturing the pre-captured X-ray image Gx1. Also, the X-ray imaging method includes step S5 of displaying the imaging assistance information J on the display 44 of the mobile terminal 4 before capturing the main captured X-ray image Gx2. Accordingly, the radiologic technologist Ra can adjust a position of the X-ray irradiator 11, its dose and the like while checking the mobile terminal 4 displaying the imaging assistance information J on the spot. Consequently, even if the radiologic technologist Ra is not experienced, it is possible to prevent repeated adjustment of the X-ray irradiator 11 after checking X-ray images Gx displayed on the display 23 arranged outside of the imaging room Rm. Also, the radiologic technologist Ra can acquire appropriate imaging assistance information J in the mobile terminal 4 by acquiring imaging assistance information J based on the pre-captured X-ray image Gx1. As these results, even if the radiologic technologist Ra is not experienced, the radiologic technologist can efficiently capture the X-ray images Gx necessary for accurate diagnosis of diseases.

Also, in the first embodiment, as described above, step S2 to step S4 of acquiring the imaging assistance information J of the X-ray imaging method include step S4 of acquiring position correction information J1 for the radiologic technologist Ra to manually correct a relative position between the X-ray irradiator 11 and the patient Pa as the imaging assistance information J based on the pre-captured X-ray image Gx1. Accordingly, since the radiologic technologist Ra can adjust the position of the X-ray irradiator 11 while checking the mobile terminal 4 displaying the position correction information J1 on the spot, it is possible to prevent the radiologic technologist Ra from repeatedly adjusting the position of the X-ray irradiator 11 after checking X-ray images Gx displayed on the display 23 arranged outside of the imaging room Rm.

Also, in the first embodiment, as described above, the imaging assistance information J includes the position correction information J1. The position correction information J1 includes information on a movement direction θ in horizontal directions and a movement distance Dt when the radiologic technologist Ra manually moves the X-ray irradiator 11 and the patient Pa relative to each other. Accordingly, since the radiologic technologist Ra can adjust the position of the X-ray irradiator 11 while checking the mobile terminal 4 indicating the movement direction θ in horizontal directions and the movement distance Dt, it is possible to accurately adjust the position of the X-ray irradiator 11.

Also, in the first embodiment, as described above, the imaging assistance information J includes the position correction information J1. The position correction information J1 is information for correcting a positional deviation between outer edges included in the bone of the patient Pa in the pre-captured X-ray image Gx1 in a direction perpendicular to the irradiation direction of the X-ray irradiator 11 based on the pre-captured X-ray image Gx1. Here, in a case in which an X-ray image Gx of the outer edges that overlap each other is captured, it is common to watch the pre-captured X-ray image Gx1 and then to perform position correction to align the outer edges with each other. Capture of such an X-ray image Gx is often very difficult even for experienced radiologic technologists Ra. Accordingly, even in the case of very difficult capture of the aforementioned X-ray image Gx, since the radiologic technologist Ra can adjust the position of the X-ray irradiator 11 while the position correction information J1 acquired in the mobile terminal 4 is displayed on the display 44 of the mobile terminal 4, the radiologic technologist can capture the X-ray images Gx necessary for accurate diagnosis of diseases, even if the radiologic technologist Ra is not experienced.

Also, in the first embodiment, as described above, step S2 to step S4 of acquiring the position correction information J1 include step S2 of finding a part of the outer edge on the X-ray irradiator 11 side and a part of the outer edge on a side opposite to the X-ray irradiator 11 side in the irradiation direction included in the bone of the patient Pa in the pre-captured X-ray image Gx1 based on the pre-captured X-ray image Gx1 to acquire the position correction information J1. Step S2 to step S4 of acquiring the position correction information J1 include step S2 of displaying the pre-captured X-ray image Gx1 on the display 44 of the mobile terminal 4 with the identification mark Ma being indicated to find the part of the outer edge on the X-ray irradiator 11 side and the part of the outer edge on the side opposite to the X-ray irradiator 11 side. Step S2 to step S4 of acquiring the position correction information J1 include step S3 of receiving determination by the radiologic technologist Ra on whether to perform position correction to align the part of the outer edge on the X-ray irradiator 11 side and the part of the outer edge on the side opposite to the X-ray irradiator 11 side with each other. Accordingly, if the radiologic technologist Ra determines that position correction is not necessary, since steps S4 and S5 are not conducted, it is possible to reduce the capture time of the X-ray image Gx by the time for steps S4 and S5.

Also, in the first embodiment, as described above, in step S1 of acquiring the pre-captured X-ray image Gx1, the pre-captured X-ray image Gx1 is acquired in the mobile terminal 4 via the network. Accordingly, since the pre-captured X-ray image Gx1 can be easily acquired, it is possible to efficiently capture the X-ray image Gx.

Also, in the first embodiment, as described above, the X-ray imaging method includes step S1 of capturing the pre-captured X-ray image Gx1 under an imaging condition in which a dose of X-rays when the pre-captured X-ray image is captured is smaller than a dose of X-rays when the main captured X-ray image Gx2 is captured. The X-ray imaging method includes step S7 of capturing, based on reception of the operation by the radiologic technologist Ra, the main captured X-ray image Gx2 under an imaging condition in which the dose of X-rays when the main captured X-ray image is captured is higher than the dose of X-rays when the pre-captured X-ray image Gx1 is captured after the pre-captured X-ray image Gx1 is captured. Accordingly, since reduction of the X-ray dose to the smaller dose of X-rays when capturing the pre-captured X-ray image Gx1 can prevent an increase of the exposure dose on the patient Pa when the X-ray imaging method is performed, it is possible to capture the X-ray images Gx necessary for accurate diagnosis of diseases while preventing the increase of the exposure dose.

Also, in the first embodiment, as described above, the X-ray imaging apparatus 100 includes the X-ray imaging apparatus 1 including the X-ray irradiator 11 irradiating the patient Pa with X-rays and the X-ray detector 15 detecting the X-rays with which the subject Pa is irradiated from the X-ray irradiator 11 and that pass through the subject. The X-ray imaging system 100 includes the mobile terminal 4 provided separately from the X-ray imaging apparatus 1. The mobile terminal 4 includes the controller 41 acquiring the imaging assistance information J for assistance in capturing the main captured X-ray image Gx2, which is captured through X-rays with which the patient is irradiated from the X-ray irradiator 11, based on the pre-captured X-ray image Gx1 that is captured through X-rays with which the patient Pa is irradiated from the X-ray irradiator 11 and that pass through the patient after capturing the pre-captured X-ray image Gx1. The mobile terminal 4 includes the display 44 displaying the imaging assistance information J acquired in the controller 41. Accordingly, the radiologic technologist Ra can adjust a position of the X-ray irradiator 11, its dose and the like while checking the mobile terminal 4 displaying the imaging assistance information J on the spot. Consequently, even if the radiologic technologist Ra is not experienced, it is possible to prevent the radiologic technologist Ra from repeatedly adjusting the X-ray irradiator 11 after checking X-ray images Gx displayed on the display 23 arranged outside of the imaging room Rm. Also, the radiologic technologist Ra can acquire appropriate imaging assistance information J in the mobile terminal 4 by acquiring imaging assistance information J based on the pre-captured X-ray image Gx1 even if the radiologic technologist Ra is not experienced. As these results, it is possible to provide the X-ray imaging system 100 capable of efficiently capturing the X-ray images Gx necessary for accurate diagnosis of diseases even if the radiologic technologist Ra is not experienced.

Also, in the first embodiment, as described above, the X-ray imaging program Pr is a program to be executed in the mobile terminal 4. The X-ray imaging program Pr causes the mobile terminal 4 to perform step S1 of acquiring the pre-captured X-ray image Gx1 through X-rays with which the subject Pa is irradiated from the X-ray irradiator 11 and that pass through the subject. The X-ray imaging program Pr causes the mobile terminal 4 to perform step S2 to step S4 of acquiring the imaging assistance information J for assistance in capturing the main captured X-ray image Gx2 captured through X-rays with which the patient is irradiated from the X-ray irradiator 11 based on the pre-captured X-ray image Gx1 after capturing the pre-captured X-ray image Gx1. The X-ray imaging program Pr causes the mobile terminal 4 to perform step S5 of displaying the imaging assistance information J on the display 44 of the mobile terminal 4 before capturing the main captured X-ray image Gx2. Accordingly, the radiologic technologist Ra can adjust a position of the X-ray irradiator 11, its dose and the like while checking the mobile terminal 4 displaying the imaging assistance information J on the spot. Consequently, even if the radiologic technologist Ra is not experienced, it is possible to prevent the radiologic technologist Ra from repeatedly adjusting the X-ray irradiator 11 after checking X-ray images Gx displayed on the display 23 arranged outside of the imaging room Rm. Also, the radiologic technologist Ra can acquire appropriate imaging assistance information J in the mobile terminal 4 by acquiring imaging assistance information J based on the pre-captured X-ray image Gx1 even if the radiologic technologist Ra is not experienced. As these results, it is possible to provide the X-ray imaging program Pr capable of efficiently capturing the X-ray images Gx necessary for accurate diagnosis of diseases even if the radiologic technologist Ra is not experienced.

### Second Embodiment

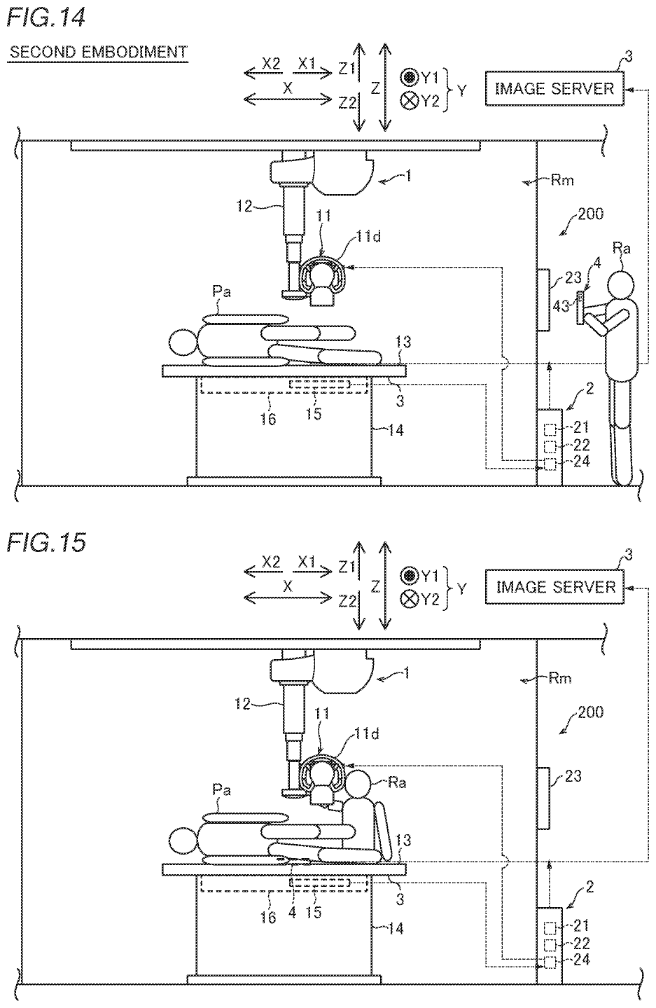

The following description describes a configuration of an X-ray imaging system 200 according to a second embodiment with reference to FIGS. 14 to 16. In the second embodiment, dissimilar to the first embodiment, the pre-captured X-ray image Gx1 is acquired by capturing the pre-captured X-ray image Gx1 that is displayed on the display 23 of the image processing apparatus 2 by using the image capturer 43 of the mobile terminal 4. Description of the same configurations in the second embodiment as those of the first embodiment is omitted.

As shown in FIGS. 14 and 15, the X-ray imaging system 200 according to the second embodiment of the present invention is a system for performing general examinations using X-rays conducted in initial diagnosis of a patient Pa who visits a medical facility such as a hospitals or clinic. Here, the patient Pa is an example of a “subject” in the claims.

Specifically, the X-ray imaging system 200 includes an X-ray imaging apparatus 1, an image processing apparatus 2, an image server 3, and a mobile terminal 4.

Here, an upward/downward direction is defined as a Z direction, and upward and downward directions are defined as Z1 and Z2 directions, respectively. In horizontal directions, longitudinal directions of the table 13 is defined as X directions, and one direction (a direction toward the image processing apparatus 2 side in FIG. 1) of the X directions and another direction (a direction toward X-ray imaging apparatus 1 in FIG. 1) of the X directions are defined as an X1 direction and an X2 direction, respectively. In horizontal directions, directions perpendicular to the X directions are defined as Y directions (shorter directions of the table 13), and one direction of the Y directions and another direction of the Y directions are defined as a Y1 direction and a Y2 direction, respectively.

### (Mobile Terminal)

The mobile terminal 4 has a function of assisting the radiologic technologist Ra in capturing X-ray images Gx of the patient Pa. The mobile terminal 4 is constructed of a tablet device.

Specifically, the mobile terminal 4 includes a controller 41, a storage 42, an image capturer 43, a display 44, a communicator 45, and a communicator 46.

### (X-Ray Imaging Program)

The mobile terminal 4 is configured to perform control, when capturing X-ray images Gx, to display imaging assistance information J so as to assist in capturing the images based on the X-ray imaging program Pr.

Specifically, as shown in FIG. 14, the controller 41 is configured to perform control to acquire the pre-captured X-ray image Gx1, which is captured through X-rays with which the patient Pa is irradiated from the X-ray irradiator 11 and that pass through the patient. Here, the controller 41 is configured to perform control to acquire the pre-captured X-ray image Gx1 that is displayed on the display 23 of the image processing apparatus 2 by capturing the pre-captured X-ray image Gx1 through the image capturer 43 provided in the mobile terminal 4. Here, the display 23 is an example of a “display apparatus” in the claims. Also, the other configuration of the second embodiment is similar to the configuration of the first embodiment, and its description is omitted.

### (X-Ray Imaging Method)

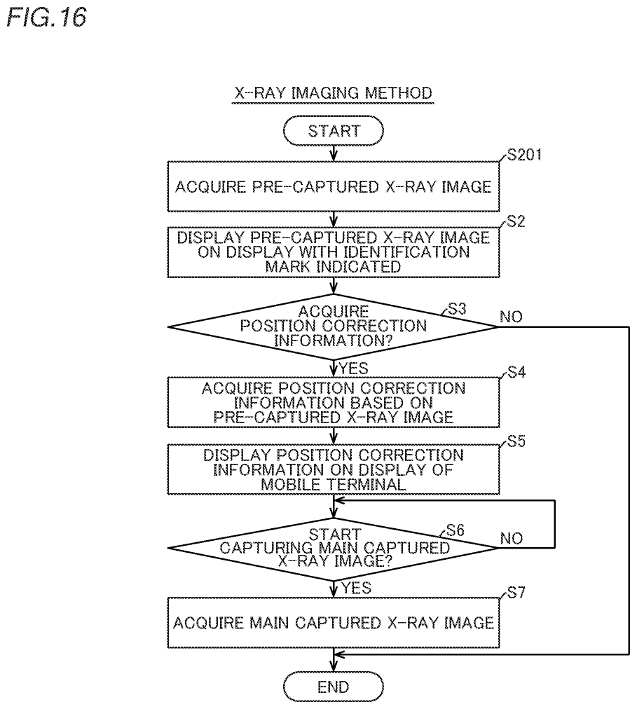

The following description describes an X-ray imaging method performed in the X-ray imaging system 100 with reference to FIG. 16.

As shown in FIG. 16, the mobile terminal 4 acquires the pre-captured X-ray image Gx1, which is captured through X-rays with which the patient Pa is irradiated from the X-ray irradiator 11 and that pass through the patient, in step S201. Specifically, the X-ray imaging apparatus 1 captures the pre-captured X-ray image Gx1 under an imaging condition in which a dose of X-rays when the pre-captured X-ray image is captured is smaller than a dose of X-rays when the main captured X-ray image Gx2 is captured. The image processing apparatus 2 generates the pre-captured X-ray image Gx1 of the patient Pa based on detection signals captured by the X-ray imaging apparatus 1. The image processing apparatus 2 displays the pre-captured X-ray image Gx1 on the display 23. The radiologic technologist Ra captures the pre-captured X-ray image Gx1 that is displayed on the display 23 of the image processing apparatus 2 by using the image capturer 43 provided in the mobile terminal 4. Accordingly, the mobile terminal 4 acquires the pre-captured X-ray image Gx1 from the image server 3 through the image capturer 43. Also, step S2 to step S7 are the same as step S2 to step S7 in the first embodiment, respectively, and their description is omitted.

### Advantages of Second Embodiment

In the second embodiment, similar to the aforementioned first embodiment above, the X-ray imaging method includes a step S201 of acquiring imaging assistance information J for assistance in capturing the main captured X-ray image Gx2 captured through X-rays with which the patient is irradiated from the X-ray irradiator 11 based on the pre-captured X-ray image Gx1 after capturing the pre-captured X-ray image Gx1 in the mobile terminal 4. Also, the X-ray imaging method includes step S5 of displaying the imaging assistance information J on the display 44 of the mobile terminal 4 before capturing the main captured X-ray image Gx2. Accordingly, even if the radiologic technologist Ra is not experienced, the radiologic technologist can efficiently capture the X-ray images Gx necessary for accurate diagnosis of diseases.

Also, in the second embodiment, as described above, in step S1 of acquiring the pre-captured X-ray image Gx1, the pre-captured X-ray image Gx1 that is displayed on the display 23 is acquired in the mobile terminal 4 by capturing the pre-captured X-ray image Gx1 through the image capturer 43 provided in the mobile terminal 4. Accordingly, even if no image server 3 is provided in the X-ray imaging system 200, it is possible to acquire the pre-captured X-ray image Gx1. The other advantages of the second embodiment are similar to the advantages of the aforementioned first embodiment.

### Third Embodiment

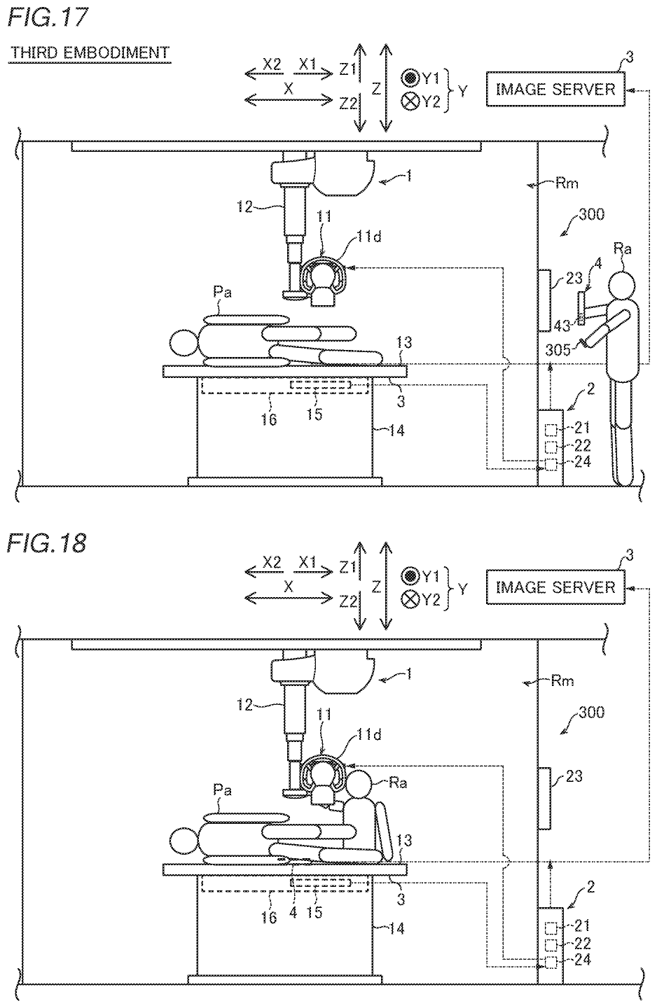

The following description describes a configuration of an X-ray imaging system 300 according to a third embodiment with reference to FIGS. 17 to 19. In the third embodiment, dissimilar to the first embodiment, the pre-captured X-ray image Gx1 is generated in the image processing apparatus 2 and stored in a portable storage medium 305, and the pre-captured X-ray image Gx1 is acquired through the communicator 46. Description of the same configurations in the third embodiment as those of the first embodiment is omitted.

As shown in FIGS. 17 and 18, the X-ray imaging system 300 according to the third embodiment of the present invention is a system for performing general examinations using X-rays conducted in initial diagnosis of a patient Pa who visits a medical facility such as a hospitals or clinic. Here, the patient Pa is an example of the “subject” in the claims.

Specifically, the X-ray imaging system 300 includes an X-ray imaging apparatus 1, an image processing apparatus 2, an image server 3, a mobile terminal 4, and the portable storage medium 305.

Here, an upward/downward direction is defined as a Z direction, and upward and downward directions are defined as Z1 and Z2 directions, respectively. In horizontal directions, longitudinal directions of the table 13 is defined as X directions, and one direction (a direction toward the image processing apparatus 2 side in FIG. 1) of the X directions and another direction (a direction toward X-ray imaging apparatus 1 in FIG. 1) of the X directions are defined as an X1 direction and an X2 direction, respectively. In horizontal directions, directions perpendicular to the X directions are defined as Y directions (shorter directions of the table 13), and one direction of the Y directions and another direction of the Y directions are defined as a Y1 direction and a Y2 direction, respectively.

### (Mobile Terminal)

The mobile terminal 4 has a function of assisting the radiologic technologist Ra in capturing X-ray images Gx of the patient Pa. The mobile terminal 4 is constructed of a tablet device.

Specifically, the mobile terminal 4 includes a controller 41, a storage 42, an image capturer 43, a display 44, a communicator 45, and a communicator 46.

### (Portable Storage Medium)

The portable storage medium 305 is a USB memory. Here, the portable storage medium 305 is a storage device with dimensions and a structure that make it easy to carry, and may be an optical disc or the like.

### (X-Ray Imaging Program)

The mobile terminal 4 is configured to perform control, when capturing X-ray images Gx, to display imaging assistance information J so as to assist in capturing the images based on the X-ray imaging program Pr.

Specifically, as shown in FIG. 17, the controller 41 is configured to perform control to acquire the pre-captured X-ray image Gx1, which is captured through X-rays with which the patient Pa is irradiated from the X-ray irradiator 11 and that pass through the patient. Here, the controller 41 is configured to perform control to acquire pre-captured X-ray image Gx1 via the portable storage medium 305. Here, the other configuration of the third embodiment is similar to the configuration of the first embodiment, and its description is omitted.

### (X-Ray Imaging Method)

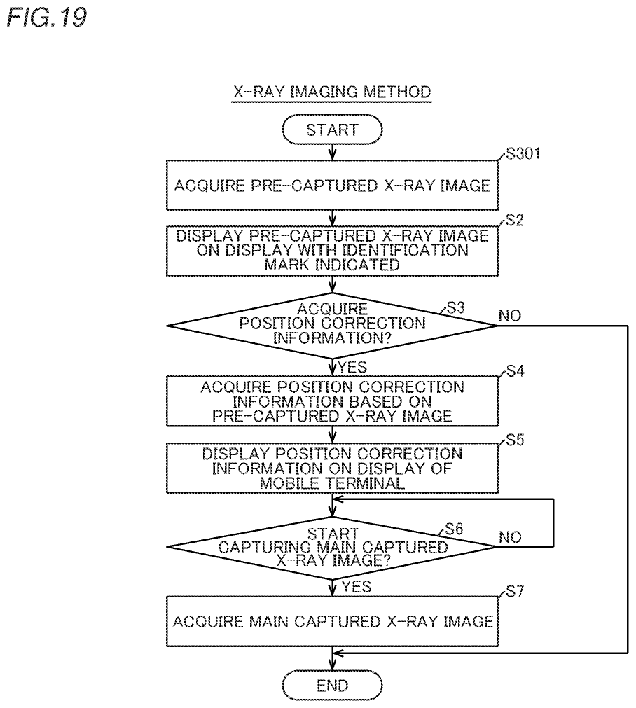

The following description describes an X-ray imaging method performed in the X-ray imaging system 300 with reference to FIG. 19.

As shown in FIG. 19, the mobile terminal 4 acquires the pre-captured X-ray image Gx1, which is captured through X-rays with which the patient Pa is irradiated from the X-ray irradiator 11 and that pass through the patient, in step S301. Specifically, the X-ray imaging apparatus 1 captures the pre-captured X-ray image Gx1 under an imaging condition in which a dose of X-rays when the pre-captured X-ray image is captured is smaller than a dose of X-rays when the main captured X-ray image Gx2 is captured. The image processing apparatus 2 generates the pre-captured X-ray image Gx1 of the patient Pa based on detection signals captured by the X-ray imaging apparatus 1. The radiologic technologist Ra connects the portable storage medium 305 to the communicator of the image processing apparatus 2 (not shown) to store the pre-captured X-ray image Gx1 generated into the portable storage medium 305. The radiologic technologist Ra connects the portable storage medium 305, which has stored the pre-captured X-ray image Gx1, to the communicator 46 of the mobile terminal 4 to store the pre-captured X-ray image Gx1 into the mobile terminal 4. Accordingly, the mobile terminal 4 acquires the pre-captured X-ray image Gx1 from the image processing apparatus 2 through the communicator 46. Also, step S2 to step S7 are the same as step S2 to step S7 in the first embodiment, respectively, and their description is omitted.

### Advantages of Third Embodiment

In the third embodiment, similar to the aforementioned first embodiment above, the X-ray imaging method includes a step S301 of acquiring imaging assistance information J for assistance in capturing the main captured X-ray image Gx2 captured through X-rays with which the patient is irradiated from the X-ray irradiator 11 based on the pre-captured X-ray image Gx1 after capturing the pre-captured X-ray image Gx1 in the mobile terminal 4. Also, the X-ray imaging method includes step S5 of displaying the imaging assistance information J on the display 44 of the mobile terminal 4 before capturing the main captured X-ray image Gx2. Accordingly, even if the radiologic technologist Ra is not experienced, the radiologic technologist can efficiently capture the X-ray images Gx necessary for accurate diagnosis of diseases.

Also, in the third embodiment, as described above, in step S1 of acquiring the pre-captured X-ray image Gx1, the pre-captured X-ray image Gx1 is acquired in the mobile terminal 4 through the portable storage medium 305. Accordingly, even if no image server 3 is provided in the X-ray imaging system 200 and no image capturer 43 is provided in the mobile terminal 4, it is possible to acquire the pre-captured X-ray image Gx1. The other advantages of the third embodiment are similar to the advantages of the aforementioned first embodiment.

### Fourth Embodiment

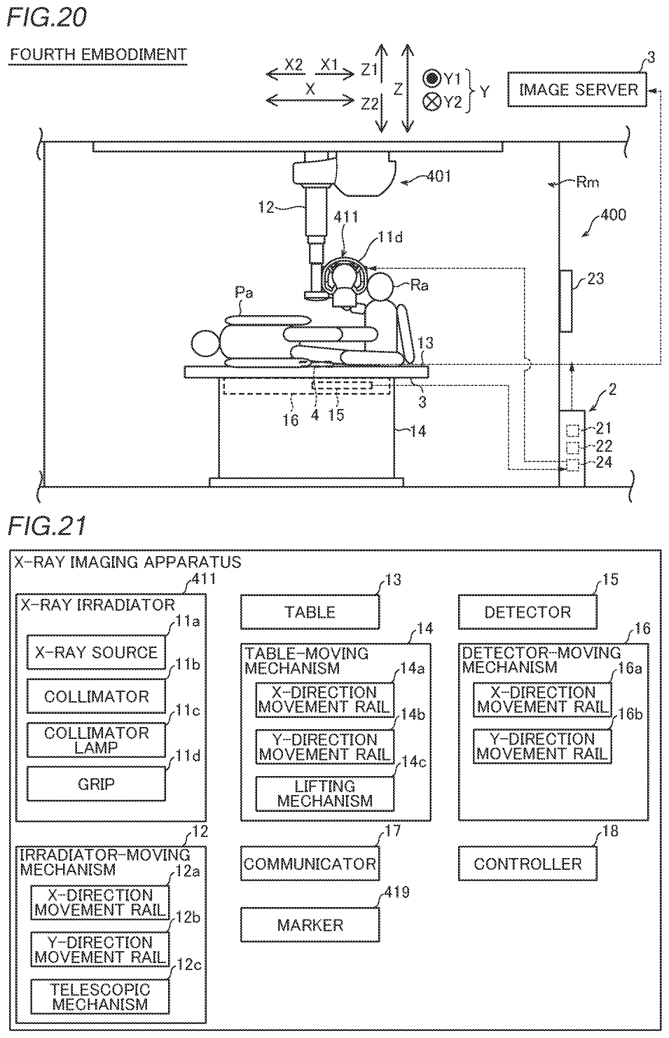

The following description describes a configuration of an X-ray imaging system 400 according to a fourth embodiment with reference to FIGS. 20 to 25. In the fourth embodiment, dissimilar to the first embodiment, the controller 41 of the mobile terminal 4 is configured to perform control to determine whether the X-ray irradiator has moved from the pre-correction position Pb to the corrected position Pf. Description of the same configurations in the fourth embodiment as those of the first embodiment is omitted.

As shown in FIGS. 20 and 21, the X-ray imaging system 400 according to the fourth embodiment of the present invention is a system for performing general examinations using X-rays conducted in initial diagnosis of a patient Pa who visits a medical facility such as a hospitals or clinic. Here, the patient Pa is an example of a “subject” in the claims.

Specifically, the X-ray imaging system 100 includes an X-ray imaging apparatus 401, an image processing apparatus 2, an image server 3, and a mobile terminal 4.

Here, an upward/downward direction is defined as a Z direction, and upward and downward directions are defined as Z1 and Z2 directions, respectively. In horizontal directions, longitudinal directions of the table 13 is defined as X directions, and one direction (a direction toward the image processing apparatus 2 side in FIG. 1) of the X directions and another direction (a direction toward X-ray imaging apparatus 1 in FIG. 1) of the X directions are defined as an X1 direction and an X2 direction, respectively. In horizontal directions, directions perpendicular to the X directions are defined as Y directions (shorter directions of the table 13), and one direction of the Y directions and another direction of the Y directions are defined as a Y1 direction and a Y2 direction, respectively.

### (X-ray Imaging Apparatus)

The X-ray imaging apparatus 401 is an apparatus for capturing X-ray images Gx of the patient Pa by using X-rays. The X-ray imaging apparatus 1 includes an X-ray irradiator 11, an irradiator-moving mechanism 12, a table 13, a table-moving mechanism 14, a detector 15, a detector-moving mechanism 16, a communicator 17, a controller 18, and a marker 419.

### <Marker>

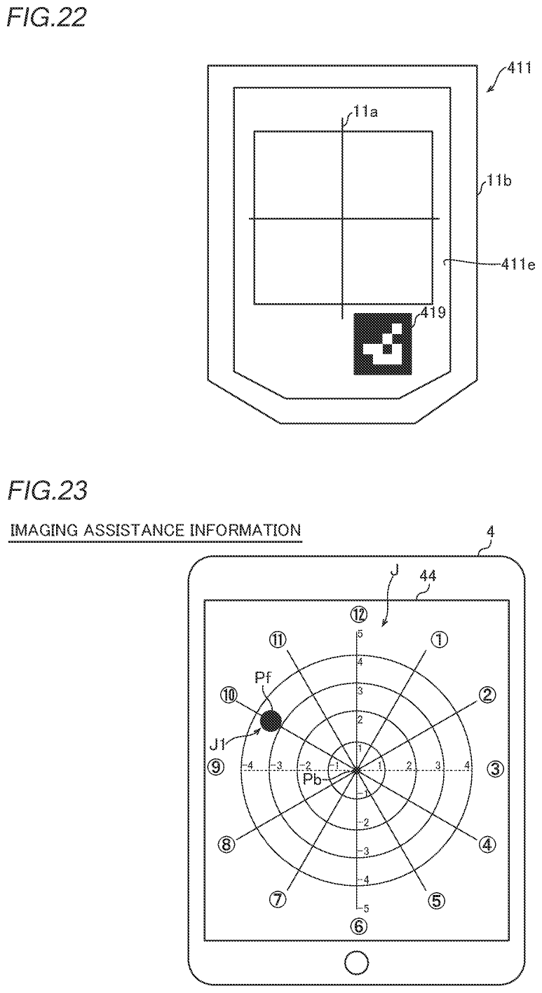

As shown in FIGS. 21 and 22, the marker 419 is a surface marker that includes an AR marker or the like. The marker 419 is provided in an irradiation window 411e allowing passage of light beams from the collimator lamp 11c and X-rays emitted for irradiation from the X-ray source 11a. The irradiation window 411e is arranged on the collimator 11b on the table 13 side.

### (Mobile Terminal)

The mobile terminal 4 has a function of assisting the radiologic technologist Ra in capturing X-ray images Gx of the patient Pa.

Specifically, the mobile terminal 4 includes a controller 41, a storage 42, an image capturer 43, a display 44, a communicator 45, and a communicator 46.

### (X-Ray Imaging Program)

The mobile terminal 4 is configured to perform control, when capturing X-ray images Gx, to display imaging assistance information J so as to assist in capturing the images based on the X-ray imaging program Pr.

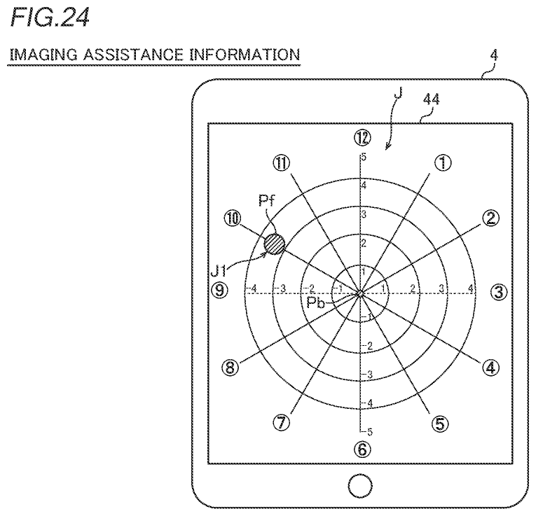

As shown in FIGS. 23 and 24, the controller 41 of the mobile terminal 4 according to the fourth embodiment is configured to perform control to acquire the imaging assistance information J for assistance in capturing the main captured X-ray image Gx2, which is captured through X-rays with which the patient is irradiated from the X-ray irradiator 11, based on an X-ray image Gx (pre-captured X-ray image Gx1) that is captured through X-rays with which the patient Pa is irradiated from the X-ray irradiator 11 and that pass through the patient, after capturing the pre-captured X-ray image Gx1. Also, the controller 41 is configured to perform control to display the imaging assistance information J acquired in the controller 41 on the display 44.

The controller 41 is configured to perform control to acquire position correction information J1 for the radiologic technologist Ra to manually correct a relative position between the X-ray irradiator 11 and the patient Pa based on the pre-captured X-ray image Gx1. In addition, it is assumed that the radiologic technologist Ra moves the X-ray irradiator 11 and the patient Pa relative to each other by manually moving the X-ray irradiator 11.

Here, the position correction information J1 is information on a pre-correction position Pb, which is shown in FIG. 23, for aligning a current position of the X-ray irradiator 11, and information on a corrected position Pf, which is shown in FIG. 24, of the X-ray irradiator 11 to which the X-ray irradiator is moved for position correction from the pre-correction position Pb.

The controller 41 is configured to control to acquire the relative position of the X-ray irradiator 411 relative to the mobile terminal 4 based on an image of the marker 419 captured by the image capturer 43 with the mobile terminal 4 being placed on the top surface of the table 13.

Specifically, the controller 41 is configured to perform control to acquire the relative position of the X-ray irradiator 411 relative to the mobile terminal 4 based on respective comparisons of a longitudinal dimension, a transverse dimension and a plan-view shape of the marker 419 that are stored in advance with a longitudinal dimension, a transverse dimension and a plan-view shape of the marker 419 in the image captured through the image capturer 43.

In other words, if a distance between the mobile terminal 4 and the marker 419 of the X-ray irradiator 411 is reduced, the longitudinal dimension and the transverse dimension of the marker 419 in the image are increased, while, if the distance between the mobile terminal 4 and the marker 419 of the X-ray irradiator 411 is increased, the longitudinal dimension and the transverse dimension of the marker 419 in the image are reduced. Accordingly, a relative height position of the X-ray irradiator 411 relative to the mobile terminal 4 is acquired. Also, if the marker 419 of the X-ray irradiator 411 is moved in a horizontal direction relative to the mobile terminal 4, the shape of the marker 419 in the image is distorted depending on the rotation angle. Accordingly, the relative positional deviation of the X-ray irradiator 411 relative to the mobile terminal 4 in the horizontal direction is acquired.

The controller 41 is configured to perform control to determine whether the X-ray irradiator 411 has arrived at the corrected position Pf based on the relative position of the X-ray irradiator 411 relative to the mobile terminal 4. The controller 41 is configured to perform control to change indication of the pre-correction position Pb and the corrected position Pf on the display 44 based on the arrival of the X-ray irradiator 411 at the corrected position Pf. In FIGS. 23 and 24, indication of the pre-correction position Pb and the corrected position Pf on the display 44 is changed from a solid black circle to a hatched circle. Here, the indication on the display 44 may be indication of only text changing from “Movement Direction: 10 o'clock direction, Movement Amount: 3 cm” to “Arrival at Target Position” and the like, or may be a combination of images, diagrams, text and the like. Also, the other configuration of the fourth embodiment is similar to the configuration of the first embodiment, and its description is omitted.

### (X-Ray Imaging Method)

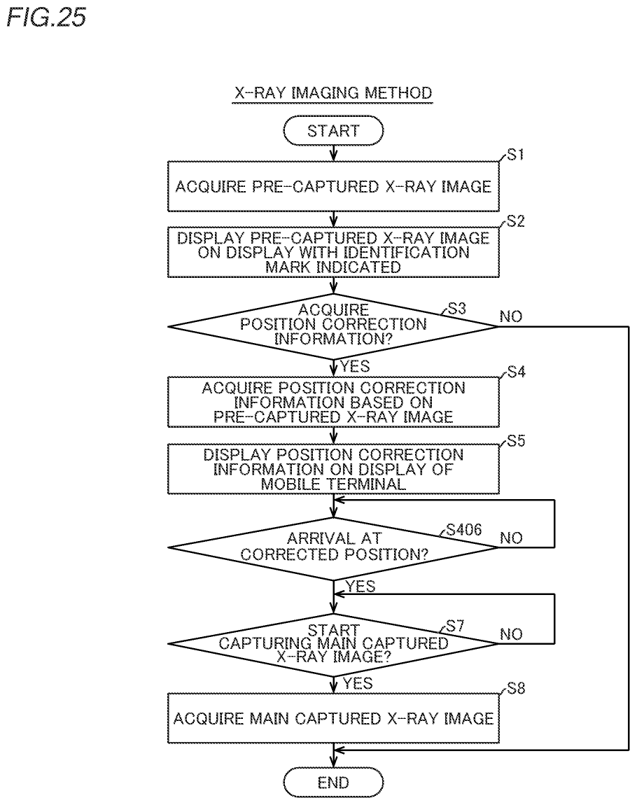

The following description describes an X-ray imaging method performed in the X-ray imaging system 400 with reference to FIG. 25.

In a flowchart shown in FIG. 25, step S1 to step S5, step S7, and step S8 are the same as step S1 to step S5, step S6, and step S7 in the flowchart shown in FIG. 13 in the first embodiment, respectively, and their description is omitted.

As shown in FIG. 25, the mobile terminal 4 determines whether the X-ray irradiator has arrived at the corrected position Pf in step S406. If the X-ray irradiator has arrived at the corrected position Pf, indication contents that represent the arrival at the corrected position Pf are displayed on the display 44, and the procedure then goes to step S7. If the X-ray irradiator has not arrived at the corrected position Pf, step S7 is repeated.

### Advantages of Fourth Embodiment

In the fourth embodiment, similar to the aforementioned first embodiment above, the X-ray imaging method includes a step S1 of acquiring imaging assistance information J for assistance in capturing the main captured X-ray image Gx2 captured through X-rays with which the patient is irradiated from the X-ray irradiator 11 based on the pre-captured X-ray image Gx1 after capturing the pre-captured X-ray image Gx1 in the mobile terminal 4. Also, the X-ray imaging method includes step S5 of displaying the imaging assistance information J on the display 44 of the mobile terminal 4 before capturing the main captured X-ray image Gx2. Accordingly, even if the radiologic technologist Ra is not experienced, the radiologic technologist can efficiently capture the X-ray images Gx necessary for accurate diagnosis of diseases.

Also, in the fourth embodiment, as described above, the imaging assistance information J includes the position correction information J1. The position correction information J1 includes the pre-correction position Pb for aligning a current position of the X-ray irradiator 11, and information on the corrected position Pf of the X-ray irradiator 11 to which the X-ray irradiator is moved for position correction from the pre-correction position Pb. Accordingly, since the radiologic technologist Ra can easily adjust the position of the X-ray irradiator 11 only by aligning the position of the X-ray irradiator 11 with the corrected position Pf indicated on the display 44 after aligning the current position of the X-ray irradiator 11 with the pre-correction position Pb indicated on the display 44, it is possible to provide easy adjustment of the position of the X-ray irradiator 11. The other advantages of the fourth embodiment are similar to the advantages of the aforementioned first embodiment.

### Modified Embodiments

Note that the embodiment disclosed this time must be considered as illustrative in all points and not restrictive. The scope of the present invention is not shown by the above description of the embodiments but by the scope of claims for patent, and all modifications (modified embodiments) within the meaning and scope equivalent to the scope of claims for patent are further included.

While the example in which the X-ray imaging system 100 (200, 300, 400) is a system for performing general examinations using X-rays conducted in initial diagnosis of a patient Pa who visits a medical facility such as a hospitals or clinic has been shown in the aforementioned first to fourth embodiments, the present invention is not limited to this. In the present invention, the X-ray imaging apparatus may be a system for other than general examinations.

While the example in which the X-ray imaging apparatus 200 (300) includes the image server 3 has been shown in the aforementioned second and third embodiments, the present invention is not limited to this. In the present invention, the X-ray imaging apparatus may include no image server 3.

While the example in which the X-ray imaging apparatus 1 (401) includes no potentiometers corresponding to positions in the XY directions (horizontal directions), a position of the Z direction (upward/downward direction), a rotational angle position about a rotation axis extending in the Z direction, and a rotational angle position of a rotation axis extending in a direction perpendicular to the Z direction when the X-ray irradiator 11 (411) is manually moved has been shown in the first to fourth embodiments, the present invention is not limited to this. In the present invention, the X-ray imaging apparatus may include potentiometers measuring positions in the horizontal directions, a position of the upward/downward direction, a rotational angle position about a rotation axis extending in the Z direction, and a rotational angle position of a rotation axis extending in a direction perpendicular to the Z direction when the X-ray irradiator is manually moved.

While the example in which the radiologic technologist Ra manually moves the X-ray irradiator 11 (411), the table 13, and the detector 15 has been shown in the first to fourth embodiments, the present invention is not limited to this. In the present invention, the X-ray irradiator, the table, and the detector may each be configured to be automatically moved by a driving source, such as a servomotor, and a stopper mechanism.

While the example in which the detector 15 is used when images are captured with the patient being lying (decubitus or lateral position) on the table 13 has been shown in the aforementioned first to fourth embodiments, the present invention is not limited to this. In the invention, the detector may be used when capturing images of a subject in a standing posture.

While the example in which the detector 15 is accommodated in an accommodation part provided in the table 13 on the Z2-direction side (downward side) to be movable in and out has been shown in the aforementioned first to fourth embodiments, the present invention is not limited to this. In the present invention, the detector may be arranged on a surface of the table on an upward side.

While the example in which the mobile terminal 4 includes the storage 42 storing the learned model Md1, the learned model Md2, and the learned model Md3 has been shown in the aforementioned first to fourth embodiments, the present invention is not limited to this. In the present invention, the learned models may be stored in a server connected to the X-ray imaging system via the network.

While the example in which the outer edge Bi of the medial condyle and the outer edge Bo of the lateral condyle of the femur of the patient Pa (subject) in the pre-captured X-ray image Gx1 are found, and respective positions of the outer edge Bi of the medial condyle and the outer edge Bo of the lateral condyle, which have been found, are corrected has been shown in the aforementioned first to fourth embodiments, the present invention is not limited to this. In the present invention, the X-ray imaging apparatus may be applied to, in a subject in the pre-captured X-ray image, position alignment in capture of image of a joint part in a shoulder, an elbow or the like in artificial bones or bones, position alignment in manipulation, position alignment of an arm in surgery (e.g., attachment of bolts to a bone), and position alignment with an imaging position at which a previous X-ray image of the subject was captured.

While the example in which the pre-captured X-ray image Gx1 is an image that is captured at the time after patient Pa (subject) has entered the imaging room Rm and before capturing the main captured X-ray image Gx2 has been shown in the aforementioned first to fourth embodiments, the present invention is not limited to this. In the present invention, the pre-captured X-ray image may be an image captured before the subject enters the imaging room.

While the example in which the imaging assistance information J is the position correction information J1 has been shown in the first to fourth embodiments, the present invention is not limited to this. In the present invention, the imaging assistance information may be dose correction information for correcting a dose of X-rays emitted for irradiation from the X-ray irradiator, or operational procedure information showing operation procedure for users when capturing the main captured X-ray image. Also, at least one of the position correction information, the dose correction information, and the operating procedure information may be displayed as the imaging assistance information to be displayed on the display of the mobile terminal. Here, the dose correction information is information for causing users to recognize an appropriate dose. Also, the operation procedure information is information including instructions on how to hold the grip, how to operate the X-ray imaging apparatus, or the like.

While the example in which the radiologic technologist Ra (user) corrects a relative position between the X-ray irradiator 11 (411) and the patient Pa (subject) by moving the X-ray irradiator 11 while grasping the grip 11d has been shown in the aforementioned first to fourth embodiments, the present invention is not limited to this. In the present invention, the user may perform correction of the relative position between the X-ray irradiator and the subject by moving the table. Also, the user may perform correction of the relative position between the X-ray irradiator and the subject by moving the subject. In this case, it is possible to determine whether the subject is moved to an appropriate position by capturing images of the subject through the image capturer of the mobile terminal before and after the user moves the subject.

While the example in which the controller 41 is configured to perform control to display the pre-captured X-ray image Gx1 on the display 44 of the mobile terminal 4 with identification marks Ma being indicated to identify the outer edge Bi found of the medial condyle, which is a part of the outer edge on the X-ray irradiator 11 (411) side, and the outer edge Bo found of the lateral condyle, which is a part of the outer edge on the side opposite to the X-ray irradiator 11 (411) side has been shown in the aforementioned first to fourth embodiments, the present invention is not limited to this. In the present invention, the controller may be configured to perform control to accept modification to a position of the identification mark by the user.

While the example in which the marker 419 is an AR marker has been shown in the aforementioned fourth embodiment, the present invention is not limited to this. In the present invention, the marker may be another surface marker, such as a QR code (registered trademark).

While the example in which the mobile terminal 4 is a tablet device has been shown in the aforementioned first to fourth embodiments, the present invention is not limited to this. In the present invention, the mobile terminal may be another mobile terminal, such as a smartphone.

While the example in which the mobile terminal 4 acquires the pre-captured X-ray image Gx1 via the network has been shown in the aforementioned first embodiment; the mobile terminal 4 acquires the pre-captured X-ray image Gx1 through the image capturer 43 has been shown in the aforementioned second embodiment; and the mobile terminal 4 acquires the pre-captured X-ray image Gx1 through the portable storage medium 305 has been shown in the aforementioned third embodiment, the present invention is not limited to this. In the present invention, the user may acquire the pre-captured X-ray images into the mobile terminal by selecting a suitable way for the X-ray imaging system from at least one of ways using the network, the image capturer, the portable storage medium, and the like.

While the example in which step S2 to step S4 of acquiring the position correction information J1 include step S3 of receiving determination by the radiologic technologist Ra (user) on whether to perform position correction to align the part of the outer edge on the X-ray irradiator 11 side and the part of the outer edge on the side opposite to the X-ray irradiator 11 side with each other has been shown in the aforementioned first to fourth embodiments, the present invention is not limited to this. In the present invention, the step of acquiring the position correction information may include a step of receiving determination of the user on whether to perform position correction to bring a relative positional relation between the part of the outer edge on the X-ray irradiator side and the part of the outer edge on the side opposite to the X-ray irradiator side to a predetermined positional relation. In this case, for example, the controller is configured to perform control to calculate, based on the deviation amount and the deviation direction found, the position correction information for correcting the relative position of the X-ray irradiator relative to the knee of the subject to a position at which the main captured X-ray image can be captured with the outer edge of the medial condyle and the outer edge of the lateral condyle being brought in a predetermined relation when capturing an image of the knee of the subject if receiving determination of the user to perform the position correction. Also, for example, the controller is configured to control to display the imaging assistance information including the position correction information on the display of the mobile terminal before capturing the main captured X-ray image. Here, the position correction to bring the relative positional relation to the predetermined positional relation is position correction other than the case in which the part of the outer edge on the X-ray irradiator side and the part of the outer edge on the side opposite to the X-ray irradiator side are aligned with each other.

While the example in which control processing of the controller 41 of the mobile terminal 4 has been described by using a flow-driven type flowchart in which processes are sequentially performed along a processing flow for ease of illustration in the aforementioned first to fourth embodiments, the present invention is not limited to this. Alternatively, in the present invention, the control processing of the controller can be executed in event-driven type processing in which the processes are executed on an event-by-event basis. In this case, the processes can be executed fully in the event-driven type processing or in combination of the event-driven type processing and flow-driven-step type processing.

### [Modes]

It is understood by those skilled in the art that the exemplary embodiments described above are specific examples of the following aspects.

### (Mode Item 1)

An X-ray imaging method includes step S1 (S201, S301) of acquiring, in a mobile terminal 4, a pre-captured X-ray image Gx1 captured through X-rays with which a subject Pa is irradiated from an X-ray irradiator 11 (411) and that pass through the subject; step S2 to step S4 of acquiring, in the mobile terminal 4, imaging assistance information J for assistance in capturing a main captured X-ray image Gx2 captured through X-rays with which the subject is irradiated from the X-ray irradiator 11 (411) based on the pre-captured X-ray image Gx1 after capturing the pre-captured X-ray image Gx1; and step S5 of displaying the imaging assistance information J on a display 44 of the mobile terminal 4 before capturing the main captured X-ray image Gx2.

### (Mode Item 2)

In the X-ray imaging method according to mode item 1, steps S2 to step S4 of acquiring the imaging assistance information include step S4 of acquiring at least one of position correction information J1 for a user Ra to manually correct a relative position between the X-ray irradiator 11 (411) and the subject Pa, dose correction information for manual correction of an dose of X-rays with which the subject is irradiated from the X-ray irradiator 11 (411), and operation procedure information indicating an operation procedure for the user Ra when capturing the main captured X-ray image Gx2 as the imaging assistance information J based on the pre-captured X-ray image Gx1.

### (Mode Item 3)

In the X-ray imaging method according to mode item 2, the imaging assistance information J includes the position correction information J1; and the position correction information J1 includes information on a movement direction θ in horizontal directions and a movement distance Dt when the user Ra manually moves the X-ray irradiator 11 (411) and the subject Pa relative to each other.

### (Mode Item 4)

In the X-ray imaging method according to mode item 2, the imaging assistance information J includes the position correction information J1; and the position correction information J1 includes information on a pre-correction position Pb for aligning a current position of the X-ray irradiator, and information on a corrected position Pf of the X-ray irradiator 11 (411) to which the X-ray irradiator is moved for position correction from the pre-correction position Pb.

### (Mode Item 5)

In the X-ray imaging method according to mode item 2, the imaging assistance information J includes the position correction information J1; and the position correction information J1 is information for correcting a positional deviation between outer edges in a direction perpendicular to an irradiation direction of the X-ray irradiator 11 (411) included in a bone(s) or an artificial joint of the subject Pa in the pre-captured X-ray image Gx1 based on the pre-captured X-ray image Gx1.

### (Mode Item 6)

In the X-ray imaging method according to mode item 5, steps S2 to step S4 of acquiring the position correction information J1 include step S2 of finding a part of the outer edge on the X-ray irradiator 11 (411) side and a part of the outer edge on a side opposite to the X-ray irradiator 11 (411) side in the irradiation direction included in the bone(s) or the artificial joint of the subject Pa in the pre-captured X-ray image Gx1 based on the pre-captured X-ray image Gx1 to acquire the position correction information J1; step S2 of displaying the pre-captured X-ray image Gx1 on the display 44 of the mobile terminal 4 with an identification mark Ma being indicated to find the part of the outer edge on the X-ray irradiator 11 (411) side and the part of the outer edge on the side opposite to the X-ray irradiator 11 (411) side; and step S3 of receiving determination of the user Ra on whether to perform position correction to align the part of the outer edge on the X-ray irradiator 11 (411) side and the part of the outer edge on the side opposite to the X-ray irradiator 11 (411) side with each other or position correction to correct a relative positional relation between the part of the outer edge on the X-ray irradiator side and the part of the outer edge on the side opposite to the X-ray irradiator side to bring the relative positional relation to a predetermined positional relation.

### (Mode Item 7)

In the X-ray imaging method according to mode item 1, in the mobile terminal 4, the pre-captured X-ray image Gx1 is acquired through network, the pre-captured X-ray image Gx1 is acquired by capturing the pre-captured X-ray image Gx1 displayed on a display apparatus 23 through an image capturer 43 provided in the mobile terminal 4, or the pre-captured X-ray image Gx1 is acquired through a portable storage medium 305 in step S1 (S201, S301) of the acquiring the pre-captured X-ray image Gx1.

### (Mode Item 8)

In the X-ray imaging method according to mode item 1, step S1 (S201, S301) of capturing the pre-captured X-ray image Gx1 under an imaging condition in which a dose of X-rays when the pre-captured X-ray image is captured is smaller than a dose of X-rays when the main captured X-ray image Gx2 is captured; and step S7 (S8) capturing the main captured X-ray image Gx2 under an imaging condition in which the dose of X-rays when the main captured X-ray image is captured is higher than the dose of X-rays when the pre-captured X-ray image Gx1 is captured after the pre-captured X-ray image Gx1 is captured are further provided.

### (Mode Item 9)

An X-ray imaging system 100 (200, 300, 400) includes an X-ray imaging apparatus 1 (401) including an X-ray irradiator 11 (411) irradiating a subject Pa with X-rays and a detector 15 detecting X-rays with which the subject is irradiated from the X-ray irradiator 11 (411) and that pass through the subject Pa; and a mobile terminal 4 provided separately from the X-ray imaging apparatus 1 (401), wherein the mobile terminal 4 includes a controller 41 acquiring imaging assistance information J for assistance in capturing a main captured X-ray image Gx2 captured through X-rays with which the subject is irradiated from the X-ray irradiator 11 (411) based on a pre-captured X-ray image Gx1 captured through X-rays with which the subject Pa is irradiated from the X-ray irradiator 11 (411) and that pass through the subject after capturing the pre-captured X-ray image Gx1, and a display 44 configured to display the imaging assistance information J acquired in the controller 41.

### (Mode Item 10)

An X-ray imaging program Pr to be executed in a mobile terminal 4, the program causing the mobile terminal 4 to perform step S1 (S201, S301) of acquiring a pre-captured X-ray image Gx1 captured through X-rays with which a subject Pa is irradiated from an X-ray irradiator 11 (411) and that pass through the subject; step S2 to step S4 of acquiring imaging assistance information J for assistance in capturing a main captured X-ray image Gx2 captured through X-rays with which the subject is irradiated from the X-ray irradiator 11 (411) based on the pre-captured X-ray image Gx1 after capturing the pre-captured X-ray image Gx1; and step S5 of displaying the imaging assistance information J on a display 44 of the mobile terminal 4 before capturing the main captured X-ray image Gx2.

### DESCRIPTION OF REFERENCE NUMERALS

1, 401; X-ray imaging apparatus
4; mobile terminal
11, 411; X-ray irradiator
15; detector
23; display (display apparatus)
41; controller
43; image capturer
44; display
100, 200, 300, 400; X-ray imaging system
305; portable storage medium
Dt; movement distance
Gx X-ray image
Gx1; pre-captured X-ray image
Gx2; main captured X-ray image
J; imaging assistance information
J1; position correction information
Ma; identification mark
Pa; patient (subject)
Pb pre-correction position
Pf; corrected position
Pr; X-ray imaging program
Ra; radiologic technologist (user)
θ; movement direction

1. An X-ray imaging method comprising:
acquiring, in a mobile terminal storing an X-ray imaging program, a pre-captured X-ray image captured through X-rays with which a subject is irradiated from an X-ray irradiator and that pass through the subject and generated by an image processing apparatus provided separately from the mobile terminal;
acquiring, in the mobile terminal, imaging assistance information for assistance in capturing a main captured X-ray image captured through X-rays with which the subject is irradiated from the X-ray irradiator based on the X-ray imaging program and the pre-captured X-ray image after capturing the pre-captured X-ray image; and
displaying the imaging assistance information on a display of the mobile terminal before capturing the main captured X-ray image.

2. The X-ray imaging method according to claim 1, wherein the acquiring the imaging assistance information includes acquiring at least one of position correction information for a user to manually correct a relative position between the X-ray irradiator and the subject, dose correction information for manual correction of an dose of X-rays with which the subject is irradiated from the X-ray irradiator, and operation procedure information indicating an operation procedure for the user when capturing the main captured X-ray image as the imaging assistance information based on the pre-captured X-ray image.

3. The X-ray imaging method according to claim 2, wherein
the imaging assistance information includes the position correction information; and
the position correction information includes information on a movement direction in horizontal directions and a movement distance when the user manually moves the X-ray irradiator and the subject relative to each other.

4. The X-ray imaging method according to claim 2, wherein
the imaging assistance information includes the position correction information; and
the position correction information includes information on a pre-correction position for aligning a current position of the X-ray irradiator, and information on a corrected position of the X-ray irradiator to which the X-ray irradiator is moved for position correction from the pre-correction position.

5. The X-ray imaging method according to claim 2, wherein
the imaging assistance information includes the position correction information; and
the position correction information is information for correcting a positional deviation between outer edges in a direction perpendicular to an irradiation direction of the X-ray irradiator included in a bone(s) or an artificial joint of the subject in the pre-captured X-ray image based on the pre-captured X-ray image.

6. An X-ray imaging method comprising:
acquiring, in a mobile terminal, a pre-captured X-ray image captured through X-rays with which a subject is irradiated from an X-ray irradiator and that pass through the subject;
acquiring, in the mobile terminal, imaging assistance information for assistance in capturing a main captured X-ray image captured through X-rays with which the subject is irradiated from the X-ray irradiator based on the pre-captured X-ray image after capturing the pre-captured X-ray image; and
displaying the imaging assistance information on a display of the mobile terminal before capturing the main captured X-ray image, wherein
the acquiring the imaging assistance information includes acquiring at least one of position correction information for a user to manually correct a relative position between the X-ray irradiator and the subject, dose correction information for manual correction of an dose of X-rays with which the subject is irradiated from the X-ray irradiator, and operation procedure information indicating an operation procedure for the user when capturing the main captured X-ray image as the imaging assistance information based on the pre-captured X-ray image,
the imaging assistance information includes the position correction information and is information for correcting a positional deviation between outer edges in a direction perpendicular to an irradiation direction of the X-ray irradiator included in a bone(s) or an artificial joint of the subject in the pre-captured X-ray image based on the pre-captured X-ray image, and
the acquiring the position correction information includes
finding a part of the outer edge on the X-ray irradiator side and a part of the outer edge on a side opposite to the X-ray irradiator side in the irradiation direction included in the bone(s) or the artificial joint of the subject in the pre-captured X-ray image based on the pre-captured X-ray image to acquire the position correction information;
displaying the pre-captured X-ray image on the display of the mobile terminal with an identification mark being indicated to find the part of the outer edge on the X-ray irradiator side and the part of the outer edge on the side opposite to the X-ray irradiator side; and
receiving determination of the user on whether to perform position correction to align the part of the outer edge on the X-ray irradiator side and the part of the outer edge on the side opposite to the X-ray irradiator side with each other or position correction to correct a relative positional relation between the part of the outer edge on the X-ray irradiator side and the part of the outer edge on the side opposite to the X-ray irradiator side to bring the relative positional relation to a predetermined positional relation.

7. The X-ray imaging method according to claim 1, wherein, in the mobile terminal, the pre-captured X-ray image is acquired through network, the pre-captured X-ray image is acquired by capturing the pre-captured X-ray image displayed on a display apparatus through an image capturer provided in the mobile terminal, or the pre-captured X-ray image is acquired through a portable storage medium in the acquiring the pre-captured X-ray image.

8. The X-ray imaging method according to claim 1 further comprising
capturing the pre-captured X-ray image under an imaging condition in which a dose of X-rays when the pre-captured X-ray image is captured is smaller than a dose of X-rays when the main captured X-ray image is captured; and
capturing the main captured X-ray image under an imaging condition in which the dose of X-rays when the main captured X-ray image is captured is higher than the dose of X-rays when the pre-captured X-ray image is captured after the pre-captured X-ray image is captured.

9. An X-ray imaging system comprising:
an X-ray imaging apparatus including an X-ray irradiator irradiating a subject with X-rays and a detector detecting X-rays with which the subject is irradiated from the X-ray irradiator and that pass through the subject; and
a mobile terminal provided separately from the X-ray imaging apparatus and an image processing apparatus, wherein
the mobile terminal includes
a storage storing an X-ray imaging program,
a controller acquiring imaging assistance information for assistance in capturing a main captured X-ray image captured through X-rays with which the subject is irradiated from the X-ray irradiator based on the X-ray imaging program and a pre-captured X-ray image captured through X-rays with which the subject is irradiated from the X-ray irradiator and that pass through the subject and generated by the image processing apparatus after capturing the pre-captured X-ray image, and
a display configured to display the imaging assistance information acquired in the controller.

10. A storage medium storing an X-ray imaging program to be executed in a mobile terminal, the program causing the mobile terminal to perform
acquiring a pre-captured X-ray image captured through X-rays with which a subject is irradiated from an X-ray irradiator and that pass through the subject and generated by an image processing apparatus separate from the mobile terminal;
acquiring imaging assistance information for assistance in capturing a main captured X-ray image captured through X-rays with which the subject is irradiated from the X-ray irradiator based on the program and the pre-captured X-ray image after capturing the pre-captured X-ray image; and
displaying the imaging assistance information on a display of the mobile terminal before capturing the main captured X-ray image.
